(19) United States
(12) Patent Application Publication
(10) Pub. No.: US 2007/0208040 A1
(43) Pub. Date: Sep. 6, 2007

(54) A2A ADENOSINE RECEPTOR ANTAGONISTS

(76) Inventors: Elfatih Elzein, Fremont, CA (US); Rao Kalla, Sunnyvale, CA (US); Jeff Zablocki, Mountain View, CA (US); Xiaofen Li, Mountain View, CA (US); Thao Perry, San Jose, CA (US); Tetsuya Kobayashi, Sunnyvale, CA (US); Eric Parkhill, South San Francisco, CA (US)

Correspondence Address: CV THERAPEUTICS, INC., 3172 PORTER DRIVE, PALO ALTO, CA, 94304, US

(21) Appl. No.: 11/681,378

(22) Filed: Mar. 2, 2007

Related U.S. Application Data
(60) Provisional application No. 60/815,745, filed on Jun. 21, 2006.
(60) Provisional application No. 60/778,821, filed on Mar. 2, 2006.

(51) Int. Cl. A61K 31/519; C07D 498/02

(52) U.S. Cl. 514/260.1; 544/278; 514/227.8; 514/234.5; 514/252.16; 544/60; 544/114

(57) ABSTRACT

The present invention relates to novel compounds that are A_{2A} adenosine receptor antagonists, and to their use in treating mammals for various disease states, such as obesity, CNS disorders, including the “movement disorders” (Parkinson's disease, Huntington's Chorea, and catelepsy), and cerebral ischemia, excitotoxicity, cognitive and physiological disorders, depression, ADHD, and drug addiction (alcohol, amphetamine, cannabinoids, cocaine, nicotine, and opioids) and to their use in the enhancement of immune response. The invention also relates to methods for the preparation of such compounds, and to pharmaceutical compositions containing them.

### CROSS REFERENCE TO RELATED APPLICATIONS

This application claims priority to U.S. Provisional Patent Applications Ser. No. 60/778,821, filed Mar. 2, 2006, and 60/815,745, filed Jun. 21, 2006, the complete disclosures of which are hereby incorporated by reference.

### FIELD OF THE INVENTION

The present invention relates to novel compounds that are A_{2A} adenosine receptor antagonists, and to their use in treating mammals for various disease states, such as obesity, CNS disorders, including the “movement disorders” (Parkinson's disease, Huntington's Chorea, and catelepsy), cerebral ischemia, excitotoxicity, cognitive and physiological disorders, depression, ADHD, and drug addiction (alcohol, amphetamine, cannabinoids, cocaine, nicotine, and opioids), and to their use in enhancing immune response. The invention also relates to methods for the preparation of such compounds, and to pharmaceutical compositions containing them.

### BACKGROUND

The effects of adenosine are transduced through adenosine receptors, which are subdivided into four general subtypes; A₁, A_{2A}, A_{2B}, and A₃, all of which modulate important physiological processes ((G. L. Stiles, K. A. Jacobson, and M. F. Jarvis, Wiley-Liss: New York, (1997); pp 29-37; V. Ralevic; G. Burnstock, G. Pharmacol. Rev. (1998) Vol. 50, 413-492). For example, stimulation of the A₁ adenosine receptors shortens the duration and decreases the amplitude of the action potential of AV nodal cells, and hence prolongs the refractory period of the AV nodal cell. Thus, stimulation of A₁ receptors provides a method of treating supraventricular tachycardias, including termination of nodal re-entrant tachycardias, and control of ventricular rate during atrial fibrillation and flutter. Stimulation of cell surface A_{2A} receptors produces dilation of the coronary resistance vessels, which phenomenon is useful for pharmacological stress imaging. A_{2B} receptors have been implicated in mast cell activation, asthma, vasodilation, regulation of cell growth, intestinal function, and modulation of neurosecretion (See Adenosine A_{2B} Receptors as Therapeutic Targets, Drug Dev Res 45:198; Feoktistov et al., Trends Pharmacol Sci 19:148-153). A₃ adenosine receptors modulate cell proliferation processes. In particular, compounds that are A₃ receptor agonists have utility in the therapeutic and/or prophylactic treatment of cancer, cardiac disease, infertility, kidney disease, and CNS disorders.

Adenosine is an important endogenous purine neuromodulator in the central nervous system. Recently, A_{2A} receptors have been demonstrated to be abundant in the basal ganglia, a region of the brain that is known to be important in motor function. It has been shown that administration of haloperidol or MPTP (N-methyl-4-phenyl-1,2,5,6-tetrahydropyridine) produces symptoms similar to Parkinson's disease in test animals, and these symptoms can be reversed by administration of an A_{2A} receptor antagonist (see, for example, “Piperazine Derivatives of [1,2,4]Triazolo[1,5-a][1,3,5]triazine as Potent and Selective Adenosine A_{2A} Receptor Antagonists”, by Chi B. Vu et al., Journal of Medicinal Chemistry, 2004).

A_{2A} receptor antagonists also possess neuroprotective properties. A_{2A} antagonists have been shown to block kainate-induced excitotoxicity in the hippocampus, to reduce ischemia-evoked glutamate and aspartate release from the cortex, and to reduce the extent of the ischemia-induced injury in rats and gerbils. Further evidence for A_{2A} receptor mediated neuroprotection arises from studies demonstrating that both cerebral infarct size and neurological deficits following transient ischemia are attenuated in A_{2A} receptor knockout mice.

Stimulation of A_{2A} adenosine receptors produces dilation of the coronary resistance vessels. Although this phenomenon is useful for pharmacological stress imaging, it is not favorable for patients who have elevated endogenous adenosine, because excessive vasodilation potentially leads to coronary steal. The phenomenon of coronary steal can cause tissue damage, because ischemia may be produced in the vascular beds fed by the artery that has lowered blood flow due to the more favorable vasodilation of healthy adjoining arteries. Accordingly, an A_{2A} antagonist will prevent the phenomenon of coronary steal.

It has been shown that adenosine signaling is implicated in drug addiction. All major drugs of abuse (opiates, cocaine, ethanol, and the like) either directly or indirectly modulate dopamine signaling in neurons, in particular those found in the nucleus accumbens, which contains high levels of A_{2A} adenosine receptors. Dependence on addictive substances has been shown to be augmented by the adenosine signaling pathway, and it has been shown that administration of an A_{2A} adenosine receptor antagonist reduces the craving for addictive substances (see. for example, “The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316), and “Adaptations in Adenosine Signaling in Drug Dependence: Therapeutic Implications”, by Stephen P. Hack and Macdonald J. Christie, Critical Review in Neurobiology, Vol. 15, 235-274 (2003)).

It has also been demonstrated that adenosine receptors, in particular the A_{2A} adenosine receptor, play a role in down regulation of inflammation in vivo (see U.S. Patent Application Publication No. 2005/0220799) by acting as a termination mechanism that limits the immune response, and consequently protect normal tissues from excess immune damage during pathogenesis of various diseases. Accordingly, inhibition of signaling through the A_{2A} adenosine receptor intensifies and prolongs the immune response in a mammal, and thus, for example, increases the efficacy of a vaccine when an A_{2A} adenosine antagonist is co-administered with a vaccine.

Accordingly, it is desired to provide compounds that are potent A_{2A} antagonists, useful in the treatment of various disease states related to modulation of the A_{2A} receptor, in particular cardiovascular diseases such as tissue damage caused by ischemia, CNS-related diseases such as Parkinson's Disease, the treatment of drug addiction, and improved immunization. Preferably, the compounds would be selective for the A_{2A} receptor, thus avoiding side effects caused by interaction with other adenosine receptors.

### SUMMARY OF THE INVENTION

It is an object of this invention to provide A_{2A} receptor antagonists. Accordingly, in a first aspect, the invention relates to compounds of Formula I:

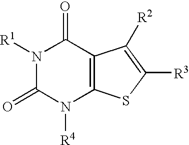

wherein
a. R¹ is hydrogen, optionally substituted C₁₋₄ alkyl, optionally substituted C₂₋₄ alkenyl, or optionally substituted C₂₋₄ alkynyl;
b. R² is hydrogen, optionally substituted C₁₋₄ alkyl, or a 5 or 6 membered optionally substituted monocyclic heterocycle containing 1, 2, 3, or 4 heteroatoms independently selected from oxygen, sulfur and nitrogen;
c. R³ is hydrogen, —C(O)NR⁵R⁶, in which R⁵ and R⁶ are independently selected from hydrogen, optionally substituted lower alkyl, or R³ is an optionally substituted 5 or 6 membered monocyclic heterocycle or heteroaryl containing 1, 2, 3, or 4 heteroatoms independently selected from oxygen, sulfur and nitrogen; or
d. R² and R³ taken together are an optionally substituted alkylene group which combine to form a 5 or 6 membered optionally substituted cyclic alkenyl ring, or a 5 or 6 membered heterocyclic ring in which the heteroatoms are oxygen or nitrogen; and
e. R⁴ is hydrogen or optionally substituted C₁₋₄ alkyl,
f. or a pharmaceutically acceptable salt, ester, prodrug, hydrate, or polymorph thereof.

In yet another embodiment of the invention, pharmaceutical formulations are provided, comprising a therapeutically effective amount of an A_{2A} receptor antagonist of Formula I, and at least one pharmaceutically acceptable carrier. The formulation is preferably for oral administration.

In a third embodiment of the invention, methods of using the compounds of Formula I in the treatment of a disease or condition in a mammal that can be treated with an A_{2A} receptor antagonist are provided. The method comprises administering to a mammal in need thereof a therapeutically effective dose of a compound of Formula I. Such diseases include, but are not limited to, obesity, Parkinson's disease, Huntington's Chorea, and catelepsy, and cerebral ischemia, excitotoxicity, and cognitive and physiological disorders, including the treatment of drug addiction. The compounds of Formula I are also useful for the inhibition of coronary vasodilation, which treatment prevents coronary steal.

In a fourth embodiment of the invention, the invention relates to compounds of Formula II: 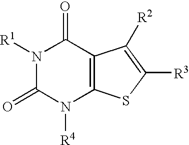
wherein
R¹ is hydrogen, optionally substituted C₁₋₄ alkyl, optionally substituted C₂₋₄ alkenyl, or optionally substituted C₂₋₄ alkynyl;
R² is hydrogen, optionally substituted C₁₋₄ alkyl, or a 5 or 6 membered optionally substituted monocyclic heterocycle containing 1, 2, 3, or 4 heteroatoms independently selected from oxygen, sulfur and nitrogen;
R³ is —COOR⁷, —C(O)R⁸, or optionally substituted lower alkyl, in which R⁷ is hydrogen or optionally substituted lower alkyl, and R⁵ is hydrogen or optionally substituted lower alkyl; and
R⁴ is hydrogen or optionally substituted C₁₋₄ alkyl.

At present, the preferred compounds for use in the invention include, but are not limited to:

6-(2-furyl)-5-methyl-3-(2-methylpropyl)-1,3-dihydrothiopheno[2,3-d]pyrimidine-2,4-dione;

3-ethyl-6-(3-furyl)-5-methyl-1,3-dihydrothiopheno[2,3-d]pyrimidine-2,4-dione;

3-ethyl-5-methyl-6-(2-thienyl)-1,3-dihydrothiopheno[2,3-d]pyrimidine-2,4-dione;

3-ethyl-6-(2-furyl)-5-methyl-1,3-dihydrothiopheno[2,3-d]pyrimidine-2,4-dione;

3-ethyl-5-methyl-6-(1,3-oxazol-2-yl)-1,3-dihydrothiopheno[2,3-d]pyrimidine-2,4-dione;

3-ethyl-5-(2-furyl)-1,3-dihydrothiopheno[2,3-d]pyrimidine-2,4-dione;

3-ethyl-5-methyl-6-(1,3-oxazolin-2-yl)-1,3-dihydrothiopheno[2,3-d]pyrimidine-2,4-dione;

(3,5-dimethyl-2,4-dioxo(1,3-dihydrothiopheno[2,3-d]pyrimidin-6-yl))-N-methylcarboxamide;

5-methyl-3-prop-2-ynyl-1,3-dihydrothiopheno[2,3-d]pyrimidine-2,4-dione;

3-ethyl-5-methyl-1,3-dihydrothiopheno[2,3-d]pyrimidine-2,4-dione;

3-ethyl-1,3,5,6,7,8-hexahydrobenzo[b]thiopheno[2,3-d]pyrimidine-2,4-dione;

3-ethyl-5-methyl-6-(3-thienyl)-1,3-dihydrothiopheno[2,3-d]pyrimidine-2,4-dione;

3,5-dimethyl-1,3-dihydrothiopheno[2,3-d]pyrimidine-2,4-dione;

N-methyl(5-methyl-2,4-dioxo-3-propyl(1,3-dihydrothiopheno[2,3-d]pyrimidin-6-yl))carboxamide;

(3-ethyl-5-methyl-2,4-dioxo(1,3-dihydrothiopheno[2,3-d]pyrimidin-6-yl))-N-methylcarboxamide;

3-ethyl-5,6-dimethyl-1,3-dihydrothiopheno[2,3-d]pyrimidine-2,4-dione;

ethyl3,5-dimethyl-2,4-dioxo-1,3-dihydrothiopheno[2,3-d]pyrimidine-6-carboxylate;

3-ethyl-5-methyl-6-[1-benzylpyrazol-4-yl]-1,3-dihydrothiopheno[2,3-d]pyrimidine-2,4-dione;

5-methyl-3-(2-methylpropyl)-1,3-dihydrothiopheno[2,3-d]pyrimidine-2,4-dione;

3-ethyl-5-methyl-6-[3-benzyl(1,2,4-oxadiazol-5-yl)]-1,3-dihydrothiopheno[2,3-d]pyrimidine-2,4-dione;

5-methyl-3-prop-2-enyl-1,3-dihydrothiopheno[2,3-d]pyrimidine-2,4-dione;

3-ethyl-5-methyl-6-pyrrol-2-yl-1,3-dihydrothiopheno[2,3-d]pyrimidine-2,4-dione;

5-methyl-3-(2-methylpropyl)-6-(1,3-oxazolin-2-yl)-1,3-dihydrothiopheno[2,3-d]pyrimidine-2,4-dione;

5-methyl-3-(2-methylpropyl)-6-(1,3-oxazol-2-yl)-1,3-dihydrothiopheno[2,3-d]pyrimidine-2,4-dione;

6-(3-furyl)-5-methyl-3-(2-methylpropyl)-1,3-dihydrothiopheno[2,3-d]pyrimidine-2,4-dione;

3-ethyl-5-methyl-6-(4-methyl(2-thienyl))-1,3-dihydrothiopheno[2,3-d]pyrimidine-2,4-dione;

3-ethyl-5-methyl-6-(3-methyl(2-thienyl))-1,3-dihydrothiopheno[2,3-d]pyrimidine-2,4-dione;

3,5-dimethyl-6-(2-thienyl)-1,3-dihydrothiopheno[2,3-d]pyrimidine-2,4-dione;

6-(2-furyl)-3,5-dimethyl-1,3-dihydrothiopheno[2,3-d]pyrimidine-2,4-dione;

3-(cyclopropylmethyl)-5-methyl-6-(2-thienyl)-1,3-dihydrothiopheno[2,3-d]pyrimidine-2,4-dione;

3-(cyclopropylmethyl)-5-methyl-6-(2-furyl)-1,3-dihydrothiopheno[2,3-d]pyrimidine-2,4-dione;

3-ethyl-5-methyl-6-(1,3-thiazol-2-yl)-1,3-dihydrothiopheno[2,3-d]pyrimidine-2,4-dione;

3-ethyl-5-methyl-6-(2-pyridyl)-1,3-dihydrothiopheno[2,3-d]pyrimidine-2,4-dione;

3-ethyl-6-(2-furyl)-1,5-dimethyl-1,3-dihydrothiopheno[2,3-d]pyrimidine-2,4-dione;

1,5-dimethyl-3-(2-methylpropyl)-6-(1,3-oxazol-2-yl)-1,3-dihydrothiopheno[2,3-d]pyrimidine-2,4-dione;

5-methyl-2,4-dioxo-3-propyl-1,3-dihydrothiopheno[2,3-d]pyrimidine-6-carboxylic acid;

N-((1R)-2-hydroxy-isopropyl)(3-ethyl-5-methyl-2,4-dioxo(1,3-dihydrothiopheno[2,3-d]pyrimidin-6-yl))carboxamide;

6-((4S)-4-methyl(1,3-oxazolin-2-yl))-3-ethyl-5-methyl-1,3-dihydrothiopheno[2,3-d]pyrimidine-2,4-dione;

1-ethyl-6-(2-furyl)-5-methyl-3-(2-methylpropyl)-1,3-dihydrothiopheno[2,3-d]pyrimidine-2,4-dione;

6-((4S)-4-methyl(1,3-oxazolin-2-yl))-5-methyl-3-propyl-1,3-dihydrothiopheno[2,3-d]pyrimidine-2,4-dione;

3-ethyl-6-(3-furyl)-1,5-dimethyl-1,3-dihydrothiopheno[2,3-d]pyrimidine-2,4-dione;

3-ethyl-1,5-dimethyl-6-(4-methyl(2-thienyl))-1,3-dihydrothiopheno[2,3-d]pyrimidine-2,4-dione;

3-ethyl-1,5-dimethyl-6-(1,3-thiazol-2-yl)-1,3-dihydrothiopheno[2,3-d]pyrimidine-2,4-dione

3-ethyl-1,5-dimethyl-6-pyrrol-2-yl-1,3-dihydrothiopheno[2,3-d]pyrimidine-2,4-dione;

3-ethyl-1,5-dimethyl-6-(2-pyridyl)-1,3-dihydrothiopheno[2,3-d]pyrimidine-2,4-dione;

6-((4S)-4-methyl(1,3-oxazolin-2-yl))-5-methyl-3-(2-methylpropyl)-1,3-dihydrothiopheno[2,3-d]pyrimidine-2,4-dione;

1,5-dimethyl-3-prop-2-ynyl-1,3-dihydrothiopheno[2,3-d]pyrimidine-2,4-dione;

6-((4S)-4-methyl(1,3-oxazolin-2-yl))-1,5-dimethyl-3-(2-methylpropyl)-1,3-dihydrothiopheno[2,3-d]pyrimidine-2,4-dione;

3-ethyl-6-(6-methoxy(3-pyridyl))-5-methyl-1,3-dihydrothiopheno [2,3-d]pyrimidine-2,4-dione;

3-ethyl-6-(2-methoxy(3-pyridyl))-5-methyl-1,3-dihydrothiopheno[2,3-d]pyrimidine-2,4-dione;

6-((4S)-4-ethyl(1,3-oxazolin-2-yl))-5-methyl-3-(2-methylpropyl)-1,3-dihydrothiopheno[2,3-d]pyrimidine-2,4-dione;

6-((5R)-5-methyl(1,3-oxazolin-2-yl))-5-methyl-3-(2-methylpropyl)-1,3-dihydrothiopheno[2,3-d]pyrimidine-2,4-dione;

6-((4S)-4-ethyl(1,3-oxazolin-2-yl))-1,5-dimethyl-3-(2-methylpropyl)-1,3-dihydrothiopheno[2,3-d]pyrimidine-2,4-dione;

6-((5R)-5-methyl(1,3-oxazolin-2-yl))-1,5-dimethyl-3-(2-methylpropyl)-1,3-dihydrothiopheno[2,3-d]pyrimidine-2,4-dione;

3-ethyl-5-(5-methyl(2-furyl))-1,3-dihydrothiopheno[2,3-d]pyrimidine-2,4-dione;

3-ethyl-1-methyl-5-(5-methyl(2-furyl))-1,3-dihydrothiopheno[2,3-d]pyrimidine-2,4-dione;

5-methyl-3-(2-methylpropyl)-6-(1,3-oxazol-4-yl)-1,3-dihydrothiopheno[2,3-d]pyrimidine-2,4-dione;

1,5-dimethyl-3-(2-propylmethyl)-6-(1,3-oxazol-4-yl)-1,3-dihydrothiopheno[1,3-d]pyrimidine-2,4-dione;

Ethyl5-methyl-2,4-dioxo-3-prop-2-ynyl-1,3-dihydrothiopheno[2,3-d]pyrimidine-6-carboxylate; and

Ethyl1,5-dimethyl-2,4-dioxo-3-prop-2-ynyl-1,3-dihydrothiopheno[2,3-d]pyrimidine-6-carboxylate.

### DETAILED DISCRIPTION OF THE INVENTION

Definitions and General Parameters

As used in the present specification, the following words and phrases are generally intended to have the meanings as set forth below, except to the extent that the context in which they are used indicates otherwise.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
1) an alkyl group as defined above, having 1, 2, 3, 4 or 5 substituents, preferably 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, aryl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
2) an alkyl group as defined above that is interrupted by 1-10 atoms independently chosen from oxygen, sulfur and NRₐ—, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, aryl, alkoxy, halogen, CF₃, amino, substituted amino, cyano, or —S(O)ₙR, in which R is alkyl, aryl, or heteroaryl and n is 0, 1 or 2; or
3) an alkyl group as defined above that has both 1, 2, 3, 4 or 5 substituents as defined above and is also interrupted by 1-10 atoms as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents, preferably 1, 2, or 3 substituents, as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, preferably 1-10 carbon atoms, more preferably 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “lower alkylene” refers to a diradical of a branched or unbranched saturated hydrocarbon chain, preferably having from 1, 2, 3, 4, 5, or 6 carbon atoms.

The term “lower alkylene” refers to a diradical of a branched or unbranched saturated hydrocarbon chain, preferably having from 1, 2, 3, 4, 5, or 6 carbon atoms.

The term“substituted alkylene” refers to:
(1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocycyl, or groups selected from carbonyl, carboxyester, carboxyamide and sulfonyl; or
(3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R—O—, where R is optionally substituted alkyl or optionally substituted cycloalkyl, or R is a group —Y-Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Preferred alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio” refers to the group R—S—, where R is as defined for alkoxy.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having 1-6, preferably 1, double-bond (vinyl). Preferred alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or prop-1-yn-3-yl, —CH₂C≡CH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, aryl, heteroaryl, heterocyclyl or where both R groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acylamino” refers to the group —NRC(O)R where each R is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, or —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Preferred aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained by the definition for the aryl or arylene substituent, such aryl or arylene groups can optionally be substituted with from 1 to 5 substituents, preferably 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, carboxyalkyl (for example, benzyloxycarbonyl), aryl, heteroaryl and heterocyclyl provided that both R groups are not hydrogen, or a group —Y-Z, in which Y is optionally substituted alkylene and Z is alkenyl, cycloalkenyl, or alkynyl, Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-oxide and N-alkoxy derivatives of nitrogen containing heteroaryl compounds, for example pyridine-N-oxide derivatives.

Unless otherwise constrained by the definition for the heteroaryl or heteroarylene substituent, such heteroaryl or heterarylene groups can be optionally substituted with 1 to 5 substituents, preferably 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclic substituent, such heterocyclic groups can be optionally substituted with 1, 2, 3, 4 or 5, and preferably 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, cycloalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” refers to a group —C(O)—.

The term “thiocarbonyl” refers to a group —C(S)—.

The term “carboxy” refers to a group —C(O)—OH.

“Optional” or “optionally” means that the subsequently described event or circumstance may or may not occur, and that the description includes instances where said event or circumstance occurs and instances in which it does not.

The term “compound of Formula I and Formula II” is intended to encompass the compounds of the invention as disclosed, and the pharmaceutically acceptable salts, pharmaceutically acceptable esters, prodrugs, hydrates and polymorphs of such compounds. Additionally, the compounds of the invention may possess one or more asymmetric centers, and can be produced as a racemic mixture or as individual enantiomers or diastereoisomers. The number of stereoisomers present in any given compound of Formula I depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound of Formula I by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“Topical administration” shall be defined as the delivery of the therapeutic agent to the surface of the wound and adjacent epithelium.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

In many cases, the compounds of this invention are capable of forming acid and/or base salts by virtue of the presence of amino and/or carboxyl groups or groups similar thereto. The term “pharmaceutically acceptable salt” refers to salts that retain the biological effectiveness and properties of the compounds of Formula I, and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “opioids” as used herein includes morphine, codeine, heroin, oxycodone (OxyContin), propoxyphene (Darvon), hydrocodone (Vicodin), and hydromorphone (Dilaudid), as well as meperidine (Demerol)

Nomenclature

The naming and numbering of the compounds of the invention is illustrated with a representative compound of Formula I in which R¹ is n-propyl, R² is methyl, and R³ is 1,3,4-oxadiazolin-2-yl, 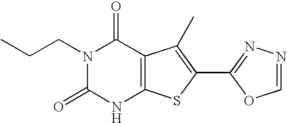
which is named: 5-methyl-6-(1,3,4-oxadiazolin-2-yl)-3-propyl-1,3-dihydrothiopheno[2,3-d]pyrimidine-2,4-dione.
Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents, and the reactions are carried out under an inert gas, preferably nitrogen.

The term “q.s.” means adding a quantity sufficient to achieve a stated function, e.g., to bring a solution to the desired volume (i.e., 100%).

### Synthesis of the Compounds of Formula I

One method of preparing compounds of Formula I is shown in Reaction Scheme I. 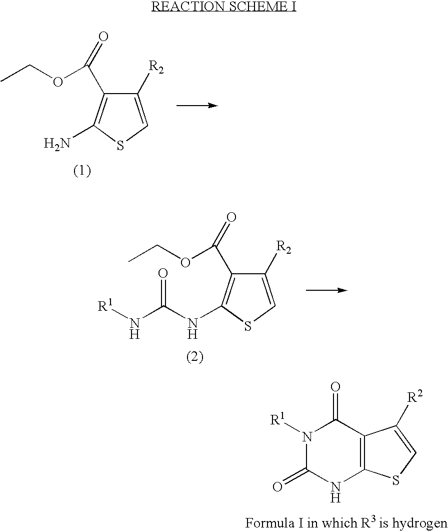
Step 1—Preparation of a Compound of Formula (2)

The compound of formula (1) is commercially available. Initially, a carboxylic acid of formula R¹CO₂H is reacted with diphenylphosphoryl azide and a base, and the resulting mixture reacted with a compound of formula (1) to provide a compound of formula (2). In general, the carboxylic acid is dissolved in an inert solvent, for example benzene or toluene, and diphenylphosphoryl azide and a tertiary organic base added, for example triethylamine, and the mixture stirred at reflux for about 1 hour. The compound of formula (1) is then added and the mixture refluxed for about 10-24 hours. When the reaction is substantially complete, the product of formula (2) is isolated by conventional means, for example by partitioning between ethyl acetate and water and purifying the product, for example by chromatography or recrystallization.

It should be noted that reaction of (1) with a carboxylic acid of formula R¹CO₂H in the presence of diphenylphosphoryl azide and a base is in effect reaction of (1) with an isocyanate of the formula R¹NCO. Many isocyanates are commercially available, or may be prepared by means well known in the art, and can be used directly in the above reaction in place of a mixture of R¹CO₂H, diphenylphosphoryl azide and a base.

Step 2—Preparation of a Compound of Formula I in which R³ is Hydrogen

The compound of formula (2) is cyclized to a compound of Formula I by reaction with an alkoxide in a protic solvent, for example sodium ethoxide in ethanol. The reaction is conducted at about room temperature for about 1-8 hours. When the reaction is substantially complete, the product of Formula I in which R³ is hydrogen is isolated and purified by conventional means.

One method of preparing a compound of Formula I in which R³ is an aromatic group is shown in Reaction Scheme II. 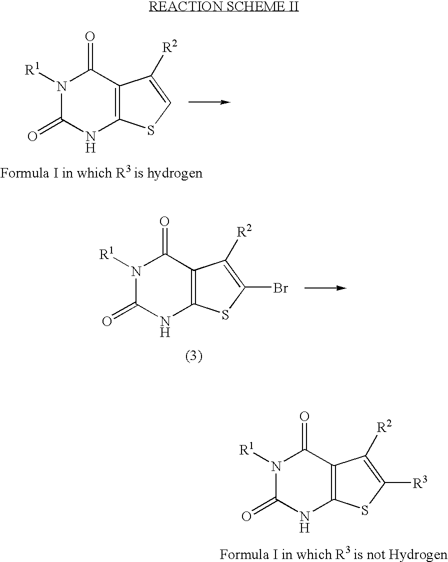
Step 1—Preparation of a Compound of Formula (3)

The compound of Formula I in which R³ is hydrogen is reacted with a brominating reagent, for example N-bromosuccinimide. In general, the compound of Formula I is dissolved in an inert solvent, for example chloroform, and cooled to about 0° C. The brominating agent is then added, and the two compounds are stirred until the reaction is complete, for example about 5-60 minutes. When the reaction is substantially complete, the product of formula (3) is isolated and purified by conventional means, for example by partitioning between methylene chloride and water. Removal of the solvent provides a compound of formula (3).

Step 2—Preparation of a Compound of Formula I

The compound of formula (3) is reacted with a compound of the formula R³B(OH)₂, which is commercially available or prepared by means well known in the art, in the presence of a catalytic amount of tetrakis(triphenylphosphine)palladium. In general, the compound of formula (3) is dissolved in an inert solvent, for example dimethoxyethane, and the compound of formula R³B(OH)₂ added, followed by the palladium catalyst and aqueous sodium carbonate solution. The reaction is conducted at a temperature of about reflux, for about 8-24 hours. When the reaction is substantially complete, the product of Formula I is isolated and purified by conventional means, for example by partitioning between ethyl acetate and water. Removal of the solvent provides a crude compound of Formula I, which may be further purified by crystallization from an inert solvent, for example ethyl acetate.

A method of preparing compounds of Formula I in which R⁴ is not hydrogen is shown in Reaction Scheme IIA. 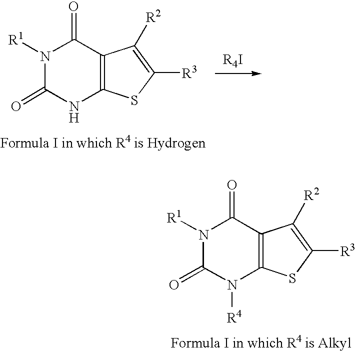
Preparation of a Compound of Formula I in which R⁴is Alkyl

The compound of Formula I in which R⁴ is hydrogen is reacted with a compound of the formula R⁴Halo, where Halo is chloro, bromo, or iodo. In general, the compound of Formula I is dissolved in a polar solvent, for example N,N-dimethylformamide, and the compound of formula R⁴Halo added, followed by a base, for example triethylamine or potassium carbonate. The reaction is conducted at a temperature of about room temperature, for about 8-24 hours. When the reaction is substantially complete, the product of Formula I is isolated and purified by conventional means, for example by removal of the solvent and purifying the residue, for example by chromatography.

An alternative method of preparing the compounds of the invention in which R³ is other than hydrogen starts from an intermediate of formula (6) or (7), the preparation of which is shown in Reaction Scheme III. 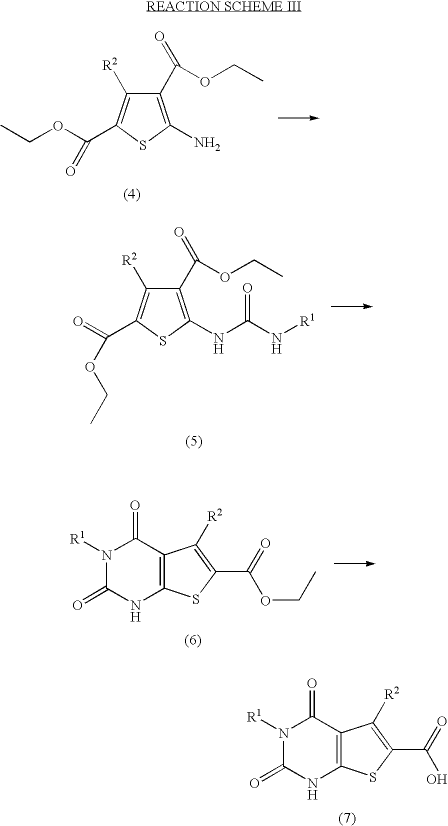
Step 1—Preparation of a Compound of Formula (5)

The compound of formula (4) is commercially available. Initially, a carboxylic acid of formula R¹CO₂H is reacted with diphenylphosphoryl azide and a base, for example triethylamine, in an inert solvent, for example toluene at reflux for about 1.5 hours. The mixture is then cooled to below 40° C., the compound of formula (4) added, and the mixture refluxed for about 24 hours. When the reaction is substantially complete, the product of formula (5) is isolated by conventional means, for example by partitioning between ethyl acetate and water and purifying the product, for example by chromatography.

Step 2—Preparation of a Compound of Formula (6)

The compound of formula (5) is cyclized to a compound of formula (6) by reaction with an alkoxide in a protic solvent, for example sodium ethoxide in ethanol. In general, the compound of formula (5) is dissolved in a protic solvent, for example ethanol, at room temperature, and an alkoxide, for example sodium ethoxide, is added. The mixture is maintained at room temperature for about 4-24 hours, for example overnight. When the reaction is substantially complete, the product of formula (6) is isolated and purified by conventional means, for example by acidifying the mixture and removing the solvent under reduced pressure.

Step 3—Preparation of a Compound of Formula (7)

The compound of formula (6) is then hydrolyzed to a compound of formula (7) by reaction with a base. In general, the compound of formula (6) is dissolved in a protic solvent, for example ethanol, in which a base is present, for example potassium hydroxide. The mixture is maintained at about 60° C. for about 4-24 hours, for example overnight. When the reaction is substantially complete, the product of formula (7) is isolated and purified by conventional means, for example by acidifying the mixture and collecting the product by filtration.

The compounds of formula (6) and (7) can then be used to prepare compounds of Formula I, examples of which are shown in the following reaction schemes. For example, to prepare a compound of Formula I in which R³ is oxazole or 1,3-oxazoline the compound of formula (7) is reacted with hydroxyethylamine, and the product cyclized. 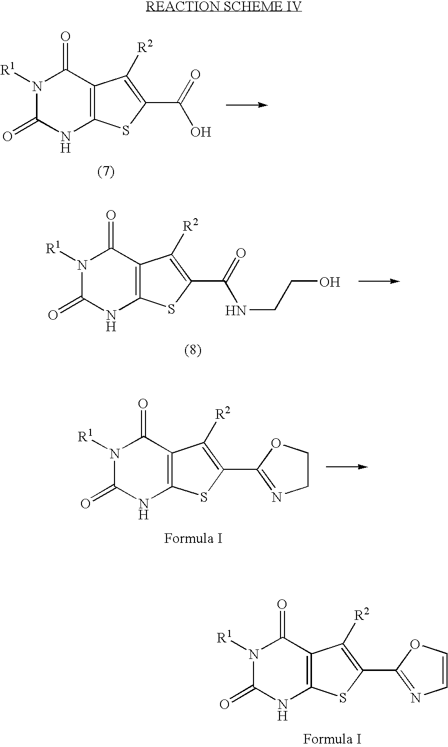
Step 1—Preparation of a Compound of Formula (8)

The compound of formula (7) is reacted with hydroxyethylamine to provide a compound of formula (8). In general, the compound of formula (7) is dissolved in an inert solvent, for example methylene chloride (and N,N-dimethylformamide of necessary to promote solubility), cooled to about 0° C., and EDCI (1-(3-dimethylaminopropyl)-3-ethylcarbodiimide hydrochloride) added, followed by HOBT (1-hydroxybenzotriazole hydrate) and a base, for example triethylamine. The mixture is maintained at about room temperature for about 12-24 hours. When the reaction is substantially complete, the product of formula (8) is isolated and purified by conventional means, for example by chromatography.

Step 2—Preparation of a Compound of Formula I

The compound of formula (8) is cyclized to provide a compound of Formula I in which R³ is 1,3-oxazoline. In general, the compound of formula (8) is dissolved in an inert solvent, for example tetrahydrofuran, and Burgess reagent [(methoxycarbonylsulfamoyl)triethylammonium hydroxide] is added. The mixture is maintained at about 70° C. for about 1-2 hours. When the reaction is substantially complete, the product of Formula I is isolated and purified by conventional means, for example by chromatography.

Step 3—Preparation of a Compound of Formula I

The compound of Formula I in which R³ is 1,3-oxazoline is converted to a compound of Formula I in which R³ is 1,3-oxazole by treatment with an oxidizing agent, for example DDQ (2,3dichloro-5,6-dicyano-1,4-benzoquinone). In general, the compound of formula (8) is dissolved in an inert solvent, for example toluene, at a temperature of about50° C., and DDQ is added. The mixture is maintained at about 70-100° C. for about 12-24 hours. When the reaction is substantially complete, the product of Formula I is isolated and purified by conventional means, for example by chromatography.

Alternatively, the carboxylic acid of formula (7) can be converted to an acid halide by reaction with oxalyl chloride, for example, which in turn is reacted with a 1-(hydroxyimino)ethylamine derivative. The product (an amino-1-azaprop-1-enyl derivative) is then cyclized conventionally to an oxadiazole derivative of Formula I, as shown in Reaction Scheme VII. 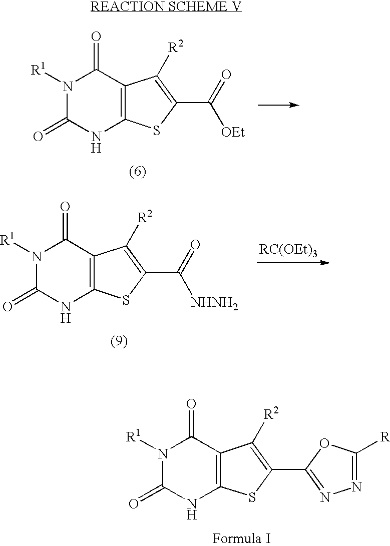
Step 1—Preparation of a Compound of Formula (9)

The compound of formula (6) is reacted with hydrazine to provide a compound of formula (9). In general, the compound of formula (6) is contacted by hydrazine monohydrate in the absence of a solvent at a temperature about 120° C. for about 1-6 hours. When the reaction is substantially complete, the product of formula (9) is isolated and purified by conventional means, for example by removal of the solvent under reduced pressure.

Step 2—Preparation of a Compound of Formula I

The compound of formula (9) is suspended in a mixture of a carboxylic acid, for example acetic acid, and a compound of the formula RC(OEt)₃, and the mixture heated in a microwave at about 120-180° C. for about 20 minutes. When the reaction is substantially complete, the product of Formula I is isolated and purified by conventional means, for example by filtration. 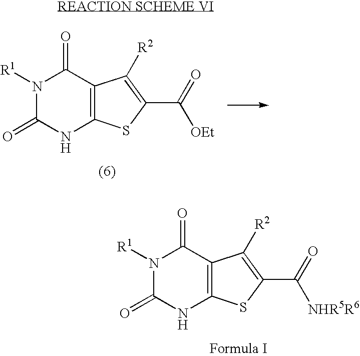
Preparation of a Compound of Formula I in which R³is an Amide

The compound of formula (6) is reacted with an amine of formula HNR⁵R⁶, in which R⁵ and R⁶ are as defined above, to provide a compound of Formula I in which R³ is an amide. In general, the compound of formula (6) is contacted with an amine of formula HNR⁵R⁶ in a protic solvent, for example methanol, at a temperature of about 60° C., preferably in a sealed tube, for about 12-24 hours. When the reaction is substantially complete, the product of Formula I in which R³ is an amide of formula —C(O)NR⁵R⁶ is isolated and purified by conventional means, for example by removal of the solvent under reduced pressure.

Alternative Preparation of a Compound of Formula I in which R³ is an Amide

The compound of formula (6) is reacted with an amine of formula HNR⁵R⁶, in which R⁵ and R⁶ are as defined above, to provide a compound of Formula I in which R³ is an amide. In general, the compound of formula (6) is contacted with an amine of formula HNR⁵R⁶ in an inert solvent, for example methylene chloride, in the presence of EDCI (1-(3-dimethylaminopropyl)-3-ethylcarbodiimide hydrochloride), at room temperature, for about 12-24 hours. When the reaction is substantially complete, the product of Formula I in which R³ is an amide of formula —C(O)NR⁵R⁶ is isolated and purified by conventional means, for example by removal of the solvent under reduced pressure and chromatography.

An alternative method of preparing compounds of Formula I in which R³ is an oxadiazole derivative is shown in Reaction Scheme VII 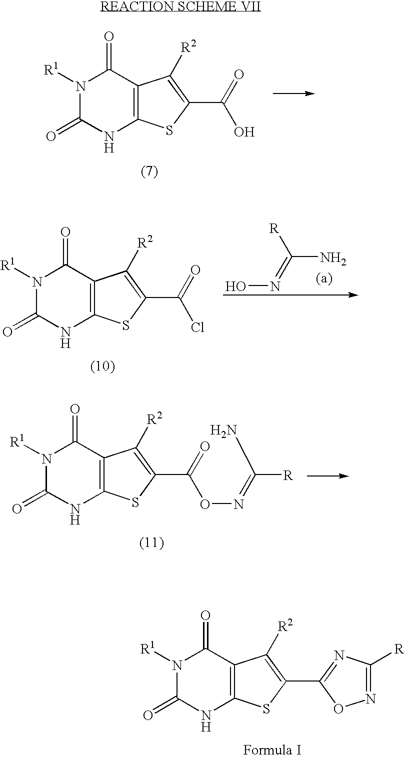
where R is any substitution.
Step 1—Preparation of a Compound of Formula (10)

The compound of formula (7) is reacted with a reagent capable of converting a carboxylic acid to an acid halide, for example thionyl chloride, oxalyl chloride, and the like, to provide a compound of formula (10). In general, the compound of formula (7) is reacted with oxalyl chloride in an inert solvent, for example tetrahydrofuran, initially at a temperature about 0° C., then at room temperature for about 12-24 hours. When the reaction is substantially complete, the product of formula (9) is isolated by conventional means, for example by removal of the solvent under reduced pressure.

Step 2—Preparation of a Compound of Formula (1)

The N-hydroxy amide oxime of formula (a) is dissolved in an inert solvent, for example tetrahydrofuran, at about 0° C., and a hindered amine added, for example N,N-diisopropylethylamine, followed by a compound of formula (10), and the mixture allowed to warm to room temperature, and maintained at that temperature for about 24 hours. When the reaction is substantially complete, the product of formula (11) is isolated and purified by conventional means, for example by preparative thin layer chromatography.

Step 3—Preparation of a Compound of Formula I

The compound of formula (11) is cyclized by heating in an inert solvent, for example toluene, at a temperature of about reflux, for about 24-72 hours. When the reaction is substantially complete, the product of formula (11) is isolated and purified by conventional means, for example by preparative thin layer chromatography.

### Utility Testing and Administration

General Utility

The compounds of the invention are A_{2A} adenosine receptor antagonists, and are effective for treating mammals for various disease states, such as obesity, CNS disorders, including the “movement disorders” (Parkinson's disease, Huntington's Chorea, and catelepsy), and cerebral ischemia, excitotoxicity, cognitive and physiological disorders, depression, ADHD, and drug addiction (alcohol, amphetamines, cannabinoids, cocaine, nicotine, and opioids).

Testing

Activity testing is conducted as described in those patents and patent applications referenced above, and in the Examples below, and by methods apparent to one skilled in the art.

Utility of the compounds of the invention for the treatment of Parkinson's disease can be tested by reversal of mouse catalepsy. In general, catalepsy is induced in mice by subcutaneous injection of haloperidol, and then the test compound is administered by oral gavage. Reversal of catalepsy demonstrates efficacy in the treatment of Parkinson's disease.

Utility of the compounds of the invention for the treatment of alcoholism can be tested by determining whether such compounds reduce the incidence of self-administration of ethanol in rats.

Utility of the compounds of the invention for the treatment of anxiety can be tested by use of the following published procedures:

Behavioral models in psychopharmacology: theoretical, industrial, and clinical perspectives, Cambridge University Press, p. 21-49 (1991); and Ann. NY Acad. Sci., 821, 332-351, (1997);

The elevated plus maze test is a model of anxiety disorder, especially for GAD, panic disorder, agoraphobia and specific phobia Psychopharmacol., 161, 314-323 (2002); Jpn. J. Psychopharmacol., 15, 125-133 (1995), and Pol. J. Pharmacol., 49, 79-84 (1997);

The social interaction test is a model of anxiety disorder, especially for GAD and social phobia, and is described in Physiol. Behav., 71, 551-557 (2000);

The vogel conflict test is a model of anxiety disorder, especially for GAD, and is described in Eur. J. Pharmacol., 463, 67-96 (2003);

The hole-board test is a model of anxiety disorder, especially for GAD, and is described in Eur. J. Pharmacol., 350, 21-29 (1998), and Pol. J. Pharmacol., 49, 79-84 (1997).

The marble burying test is a model of anxiety disorder, especially for OCD, and is described in Jpn. J. Pharmacol. 68, 65-70 (1995); and

Learned helplessness is a model of anxiety disorder, especially for PTSD, and is described in Green et al., Behavioral models in psychopharmacology: theoretical, industrial, and clinical perspectives, Cambridge University Press, p. 21-49 (1991); and Ann. NY Acad. Sci., 821, 332-351, (1997).

Pharmaceutical Compositions

The compounds of Formula I are usually administered in the form of pharmaceutical compositions. This invention therefore provides pharmaceutical compositions that contain, as the active ingredient, one or more of the compounds of Formula I, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. The compounds of Formula I may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G.S. Banker & C.T. Rhodes, Eds.).

Administration

The compounds of Formula I may be administered in either single or multiple doses by any of the accepted modes of administration of agents having similar utilities, for example as described in those patents and patent applications incorporated by reference, including buccal, intranasal, intra-arterial injection, intravenously, intraperitoneally, parenterally, intramuscularly, subcutaneously, orally, or as an inhalant.

Oral administration is the preferred route for administration of the compounds of Formula I. Administration may be via capsule or enteric coated tablets, or the like. In making the pharmaceutical compositions that include at least one compound of Formula I, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Adenosine A_{2A} receptor antagonists such as the compounds of Formula I are effective over a wide dosage range and is generally administered in a pharmaceutically effective amount. Typically, for oral administration, each dosage unit contains from 1 mg to 2 g of an adenosine A_{2A} receptor antagonist, more commonly from 1 to 700 mg, and for parenteral administration, from 1 to 700 mg of an adenosine A_{2A} receptor antagonist, more commonly about 2 to 200 mg. It will be understood, however, that the amount of the adenosine A_{2A} receptor antagonist actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation of a Compound of Formula (2)

A. Preparation of a Compound of Formula (2) in Which R¹ is Ethyl and R² is Methyl

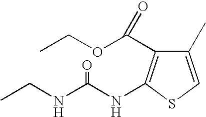

A mixture of propionic acid (3.8 ml, 50.64 mmol), diphenylphosphoryl azide (10.9 ml, 50.64 mmol), and triethylamine (7.1 ml, 50.64 mmol) in toluene (30 ml) was refluxed for 1 hour. After cooling to room temperature, ethyl2-amino-4-methylthiophene-3-carboxylate (3.13 g, 16.88 mmol) was added, and the mixture refluxed for 18 hours. The product was partitioned between ethyl acetate and water, the organic layer washed with brine, dried over sodium sulfate, and solvent removed under reduced pressure. The residue was chromatographed on silica gel, eluting with ethyl acetate/hexane 1:1, to provide ethyl4-methyl-2-[(methylamino)carbonylamino]-thiophene-3-carboxylate as pink crystals.

B. Preparation of Compounds of Formula (2) Varying R¹ and R²

Similarly, following the procedure of Example 1A above, but optionally substituting other compounds of formula (1) for ethyl2-amino-4-methylthiophene-3-carboxylate, and optionally substituting other carboxylic acids of formula R¹CO₂H for acetic acid, the following compounds of formula (2) were prepared:
ethyl4-methyl-2-[(ethylamino)carbonylamino]thiophene-3-carboxylate;
ethyl4-methyl-2-[(propylamino)carbonylamino]thiophene-3-carboxylate;
ethyl4-methyl-2-{[(methylethyl)amino]carbonylamino}thiophene-3-carboxylate;
ethyl4-methyl-2-{[(2-methylpropyl)amino]carbonylamino}thiophene-3-carboxylate;
ethyl2-[(butylamino)carbonylamino]-4-methylthiophene-3-carboxylate;
ethyl2-{[(cyclopropylmethyl)amino]carbonylamino}-4-methylthiophene-3-carboxylate;
ethyl4-methyl-2-[(phenylamino)carbonylamino]thiophene-3-carboxylate;
ethyl4-methyl-2-[(2-thienylamino)carbonylamino]thiophene-3-carboxylate;
ethyl4-methyl-2-({[(4-methylphenyl)methyl]amino}carbonylamino)thiophene-3-carboxylate;
ethyl2-[(ethylamino)carbonylamino]-4-phenylthiophene-3-carboxylate;
ethyl2-[(ethylamino)carbonylamino]-4-(2-furyl)thiophene-3-carboxylate;
ethyl2-[(ethylamino)carbonylamino]-4-(2-thienyl)thiophene-3-carboxylate;
ethyl2-[(ethylamino)carbonylamino]-4-(4-phenylphenyl)thiophene-3-carboxylate;
ethyl2-[(ethylamino)carbonylamino]-4-(methylethyl)thiophene-3-carboxylate;
ethyl4-methyl-2-[(prop-2-ylamino)carbonylamino]thiophene-3-carboxylate;
ethyl4-methyl-2-[(prop-2-enylamino)carbonylamino]thiophene-3-carboxylate;
ethyl4-methyl-2-{[(2-methylpropyl)amino]carbonylamino}thiophene-3-carboxylate; and
ethyl2-[(ethylamino)carbonylamino]-4,5,6,7-tetrahydrobenzo[b[thiophene-3-carboxylate.
   C. Preparation of Compounds of Formula (2) Varying R¹ and R²

Similarly, following the procedure of Example IA above, but optionally substituting other compounds of formula (1) for ethyl2-amino-4-methylthiophene-3-carboxylate, and optionally substituting other carboxylic acids of formula R¹CO₂H for acetic acid substituting carboxylic acids of formula R¹CO₂H, where R¹ is as defined above for acetic acid, other compounds of formula (2) are prepared.

### EXAMPLE 2

### Preparation of a Compound of Formula I

A. Preparation of a Compound of Formula I in Which R¹ is Ethyl, R² is Methyl, and R³ is Hydrogen

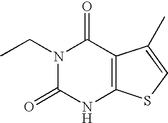

To a suspension of ethyl4-methyl-2-[(ethylamino)carbonylamino]thiophene-3-carboxylate (3.44 g, 13.46 mmol) in ethanol (10 ml) was added a solution of sodium ethoxide (2M in ethanol, 10 ml, 20 mmol) at room temperature, and the mixture stirred at room temperature for 2 hours. Ice was then added to the reaction mixture, which was then cooled in an ice bath and acidified with concentrated hydrochloric acid to a pH of less than 1. Water (70 ml) was then added, and the resulting solid filtered off, washed with water, and then dried under reduced pressure to provide 3-ethyl-5-methyl-1,3-dihydrothiopheno[2,3-d]pyrimidine-2,4-dione.

B. Preparation of a Compound of Formula I in which R³ is Hydrogen, Varying R¹ and R²

Similarly, following the procedure of Example 2A above, but substituting other compounds of formula (2) for ethyl4-methyl-2-[(methylamino)carbonylamino]-thiophene-3-carboxylate, the following compounds of Formula I in which R³ is hydrogen were prepared.
3,5-dimethyl-1,3-dihydrothiopheno[2,3-d]pyrimidine-2,4-dione;
3-propyl-5-methyl-1,3-dihydrothiopheno[2,3-d]pyrimidine-2,4-dione;
3-methylethyl-5-methyl-1,3-dihydrothiopheno[2,3-d]pyrimidine-2,4-dione;
3-(2-methylpropyl)-5-methyl-1,3-dihydrothiopheno[2,3-d]pyrimidine-2,4-dione;
3-butyl-5-methyl-1,3-dihydrothiopheno[2,3-d]pyrimidine-2,4-dione;
3-cyclopropylmethyl-5-methyl-1,3-dihydrothiopheno[2,3-d]pyrimidine-2,4-dione;
3-phenyl-5-methyl-1,3-dihydrothiopheno[2,3-d]pyrimidine-2,4-dione;
3-(2-thienyl)-5-methyl-1,3-dihydrothiopheno[2,3-d]pyrimidine-2,4-dione;
3-(4-methylphenyl)-5-methyl-1,3-dihydrothiopheno[2,3-d]pyrimidine-2,4-dione;
3-ethyl-5-(methylethyl)-1,3-dihydrothiopheno[2,3-d]pyrimidine-2,4-dione;
3-ethyl-5-phenyl-1,3-dihydrothiopheno[2,3-d]pyrimidine-2,4-dione;
3-ethyl-5-(4-phenylphenyl)-1,3-dihydrothiopheno[2,3-d]pyrimidine-2,4-dione;
1,5-dimethyl-3-prop-2-ynyl-1,3-dihydrothiopheno[2,3-d]pyrimidine-2,4-dione;
3-ethyl-5-(2-furyl)-1,3-dihydrothiopheno[2,3-d]pyrimidine-2,4-dione;
3-ethyl-5-(2-thienyl)-1,3-dihydrothiopheno[2,3-d]pyrimidine-2,4-dione;
5-methyl-3-prop-2-ynyl-1,3-dihydrothiopheno[2,3-d]pyrimidine-2,4-dione;
5-methyl-3-prop-2-enyl-1,3-dihydrothiopheno[2,3-d]pyrimidine-2,4-dione;
5-methyl-3-(2-methylpropyl)-1,3-dihydrothiopheno[2,3-d]pyrimidine-2,4-dione;
3-ethyl-1,3,5,6,7,8-hexahydrobenzo[b]thiopheno[2,3-d]pyrimidine-2,4-dione;
3-ethyl-5-(5-methyl(2-furyl))-1,3-dihydrothiopheno[2,3-d]pyrimidine-2,4-dione;
3-ethyl-1-methyl-5-(5-methyl(2-furyl))-1,3-dihydrothiopheno[2,3-d]pyrimidine-2,4-dione;
5-methyl-3-prop-2-enyl-1,3-dihydrothiopheno[2,3-d]pyrimidine-2,4-dione; and
5-methyl-3-prop-2-ynyl-1,3-dihydrothiopheno[2,3-d]pyrimidine-2,4-dione.
   C. Preparation of Compounds of Formula I in Which R³ is not Hydrogen

Similarly, following the procedure of Example 2A above, but substituting other compounds of formula (2) for ethyl4-methyl-2-[(methylamino)carbonylamino]-thiophene-3-carboxylate, the following compounds of Formula I in which R3 is not hydrogen were prepared:

3-ethyl-5,6-dimethyl-1,3-dihydrothiopheno[2,3-d]pyrimidine-2,4-dione; and

6-acetyl-3-ethyl-5-methyl-1,3-dihydrothiopheno[2,3-d]pyrimidine-2,4-dione.

### EXAMPLE 3

### Preparation of a Compound of Formula (3)

A. Preparation of a Compound of Formula (3) in Which R¹ is Ethyl and R² is Methyl

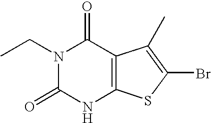

A suspension of 3-ethyl-5-methyl-1,3-dihydrothiopheno[2,3-d]pyrimidine-2,4-dione (2.58 g, 12.27 mmol) in chloroform (60 ml) was cooled to 0° C., and N-bromosuccinimide (2.18 g, 12.27 mmol) added in portions over 15 minutes with stirring. The mixture was stirred for 30 minutes, then methanol (10 ml) was added, causing the solid to go into solution. The solution was extracted with water (30 ml), and the aqueous layer washed with methylene chloride (2×50 ml). After combining the organic layers, a solid formed, and thus a further 20 ml of methanol was added to dissolve the solid. The solution was dried over sodium sulfate, filtered, and solvent removed from the filtrate under reduced pressure, providing 6-bromo-3-ethyl-5-methyl-1,3-dihydrothiopheno[2,3-d]pyrimidine-2,4-dione as brown crystals. Crystallization of this solid from ethyl acetate (30 ml) provided a light brown solid. Chromatography of the residue on silica gel, eluting with ethyl acetate/hexane 1:1 provided further product.

B. Preparation of a Compound of Formula (3), varying R¹ and R²

Similarly, following the procedure of Example 3A above, but substituting other compounds of Formula I in which R³ is hydrogen for 3-ethyl-5-methyl-1,3-dihydrothiopheno[2,3-d]pyrimidine-2,4-dione, the following compounds of formula (3) were prepared.
6-bromo-3-ethyl-5-methyl-1,3-dihydrothiopheno[2,3-d]pyrimidine-2,4-dione;
6-bromo-3-propyl-5-methyl-1,3-dihydrothiopheno[2,3-d]pyrimidine-2,4-dione;
6-bromo-3-methylethyl-5-methyl-1,3-dihydrothiopheno[2,3-d]pyrimidine-2,4-dione;
6-bromo-3-(2-methylpropyl)-5-methyl-1,3-dihydrothiopheno[2,3-d]pyrimidine-2,4-dione;
6-bromo-3-butyl-5-methyl-1,3-dihydrothiopheno[2,3-d]pyrimidine-2,4-dione;
6-bromo-3-cyclopropylmethyl-5-methyl-1,3-dihydrothiopheno[2,3-d]pyrimidine-2,4-dione;
6-bromo-3-phenyl-5-methyl-1,3-dihydrothiopheno[2,3-d]pyrimidine-2,4-dione;
6-bromo-3-(2-thienyl)-5-methyl-1,3-dihydrothiopheno[2,3-d]pyrimidine-2,4-dione;
6-bromo-3-(4-methylphenyl)-5-methyl-1,3-dihydrothiopheno[2,3-d]pyrimidine-2,4-dione; and
6-bromo-3-ethyl-5-(methylethyl)-1,3-dihydrothiopheno [2,3-d]pyrimidine-2,4-dione.
   C. Preparation of Compounds of Formula (3) Varying R¹ and R²

Similarly, following the procedure of Example 3A above, but substituting other compounds of Formula I in which R³ is hydrogen for 3-ethyl-5-methyl-1,3-dihydrothiopheno[2,3-d]pyrimidine-2,4-dione, other compounds of formula (3) are prepared, for example:
6-bromo-3-ethyl-5-phenyl-1,3-dihydrothiopheno[2,3-d]pyrimidine-2,4-dione;
6-bromo-3-ethyl-5-(4-phenylphenyl)-1,3-dihydrothiopheno[2,3-d]pyrimidine-2,4-dione;
6-bromo-3-ethyl-5-(2-furyl)-1,3-dihydrothiopheno[2,3-d]pyrimidine-2,4-dione; and
6-bromo-3-ethyl-5-(2-thienyl)-1,3-dihydrothiopheno[2,3-d]pyrimidine-2,4-dione.

### EXAMPLE 4

### Preparation of a Compound of Formula I

A. Preparation of a Compound of Formula I in which R¹ is Ethyl, R² is Methyl, and R⁴ is Hydrogen

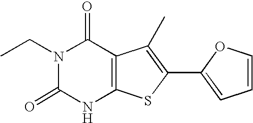

A mixture of 6-bromo-3-ethyl-5-methyl-1,3-dihydrothiopheno[2,3-d]pyrimidine-2,4-dione (97.6 mg 0.338 mmol), 2-furanboronic acid (94.5 mg, 0.845 mmol), tetrakis(triphenylphosphine)palladium (19.5 mg, 0.0169 mmol), dimethoxyethane (4 ml) and aqueous sodium carbonate (1 ml) was prepared, and the mixture refluxed under nitrogen for 15 hours. The product was filtered through Celite (3 g), washing the celite with ethyl acetate (70 ml). The filtrate was washed with water (30 ml), followed by brine (30 ml) and dried over sodium sulfate. Solvent was removed from the dried solution under reduced pressure to give crude 3-ethyl-6-(2-furyl)-5-methyl-1,3-dihydrothiopheno-[2,3-d]pyrimidine-2,4-dione as a brown oil. Crystallization from ethyl acetate (1 ml) gave the product as light brown crystals.

¹H NMR: (400 MHz, CDCl₃)

1.30 (3 H, t, J=7.0 Hz, —CH₂CH₃), 2.66 (3H, s, CH₃(C5)), 4.10 (2H, q, J=7.0 Hz, —CH₂CH₃), 6.49 (1H, d, J=2.7 Hz, —CH(furyl-C2)), 6.51 (1H, dd, J=2.7, 2.0 Hz, —CH(furyl-C4)), 7.44 (1H, d, J=2.0 Hz, —CH(furyl-C5)), 10.55 (1H, br s, NH);

MS (EI): 276 (M⁺)

B. Preparation of a Compound of Formula I, Varying R¹, R² and R³

Similarly, following the procedure of Example 4A above, but optionally substituting other compounds of formula (3) for 6-bromo-3-ethyl-5-methyl-1,3-dihydrothiopheno[2,3-d]pyrimidine-2,4-dione, and optionally substituting other boronic acid derivatives for 2-furanboronic acid, the following compounds of Formula I were prepared:
3-ethyl-5-methyl-6-(2-thienyl)-1,3-dihydrothiopheno[2,3-d]pyrimidine-2,4-dione;
3-ethyl-5-methyl-6-[4-(phenylmethoxy)phenyl]-1,3-dihydrothiopheno[2,3-d]pyrimidine-2,4-dione;
3-ethyl-5-methyl-6-[1-benzylpyrazol-4-yl]-1,3-dihydrothiopheno[2,3-d]pyrimidine-2,4-dione;
3-ethyl-6-(3-furyl)-5-methyl-1,3-dihydrothiopheno[2,3-d]pyrimidine-2,4-dione;
3-ethyl-5-methyl-6-(3-thienyl)-1,3-dihydrothiopheno[2,3-d]pyrimidine-2,4-dione;
6-(2-furyl)-5-methyl-3-(2-methylpropyl)-1,3-dihydrothiopheno[2,3-d]pyrimidine-2,4-dione;
6-(2-thienyl)-5-methyl-3-(2-methylpropyl)-1,3-dihydrothiopheno[2,3-d]pyrimidine-2,4-dione;
5-methyl-3-ethyl-6-phenyl-1,3-dihydrothiopheno[2,3-d]pyrimidine-2,4-dione;
5-methyl-3-ethyl-6-[3-benzyl(1,2,4-oxadiazol-5-yl)]-1,3-dihydrothiopheno[2,3-d]pyrimidine-2,4-dione;
5-methyl-3-(2-methylpropyl)-6-[1-benzylpyrazol-4-yl]-1,3-dihydrothiopheno[2,3-d]pyrimidine-2,4-dione;
6-(3-furyl)-5-methyl-3-(2-methylpropyl)-1,3-dihydrothiopheno[2,3-d]pyrimidine-2,4-dione;
3-ethyl-5-methyl-6-(5-methyl(2-thienyl))-1,3-dihydrothiopheno[2,3-d]pyrimidine-2,4-dione;
3-ethyl-5-methyl-6-(4-methyl(2-thienyl))-1,3-dihydrothiopheno[2,3-d]pyrimidine-2,4-dione;
3-ethyl-5-methyl-6-(3-methyl(2-thienyl))-1,3-dihydrothiopheno[2,3-d]pyrimidine-2,4-dione;
3-ethyl-5-methyl-6-(5-chloro(2-thienyl))-1,3-dihydrothiopheno[2,3-d]pyrimidine-2,4-dione;
3,5-dimethyl-6-(2-thienyl)-1,3-dihydrothiopheno[2,3-d]pyrimidine-2,4-dione;
6-(2-furyl)-3,5-dimethyl-1,3-dihydrothiopheno[2,3-d]pyrimidine-2,4-dione;
3-(cyclopropylmethyl)-5-methyl-6-(2-thienyl)-1,3-dihydrothiopheno[2,3-d]pyrimidine-2,4-dione;
3-(cyclopropylmethyl)-5-methyl-6-(2-furyl)-1,3-dihydrothiopheno[2,3-d]pyrimidine-2,4-dione;
1-ethyl-6-(2-furyl)-5-methyl-3-(2-methylpropyl)-1,3-dihydrothiopheno[2,3-d]pyrimidine-2,4-dione;
3-ethyl-6-methyl-1,3-dihydrothiopheno[2,3-d]pyrimidine-2,4-dione;
1,5-dimethyl-6-(2-methyl(1,3-thiazol-4-yl))-3-(2-methylpropyl)-1,3-dihydrothiopheno[2,3-d]pyrimidine-2,4-dione;
5-methyl-3-(2-methylpropyl)-6-(1,3-oxazol-4-yl)-1,3-dihydrothiopheno[2,3-d]pyrimidine-2,4-dione; and
3-ethyl-5-methyl-6-(4-pyridyl)-1,3-dihydrothiopheno[2,3-d]pyrimidine-2,4-dione.
   C. Preparation of Compounds of Formula I Varying R¹, R², R³, and R⁴

Similarly, following the procedure of Example 4A above, but optionally substituting other compounds of formula (3) for 6-bromo-3-ethyl-5-methyl-1,3-dihydrothiopheno[2,3-d]pyrimidine-2,4-dione, and optionally substituting other boronic acid derivatives for 2-furanboronic acid, the following compounds of Formula I were prepared:

3-ethyl-1,5-dimethyl-6-pyrrol-2-yl-1,3-dihydrothiopheno[2,3-d]pyrimidine-2,4-dione.

D. Preparation of Compounds of Formula I Varying R¹, R², R³, and R⁴

Optionally substituting other compounds of formula (3) above for 6-bromo-3-ethyl-5-methyl-1,3-dihydrothiopheno[2,3-d]pyrimidine-2,4-dione the following compounds of Formula I were prepared using Stille reaction conditions {Pd(PPh₃)₄ [10 mol %], the appropriate RSnBu₃, toluene, microwave 160° C., 1 h}:
3-ethyl-5-methyl-6-(1,3-thiazol-2-yl)-1,3-dihydrothiopheno[2,3-d]pyrimidine-2,4-dione; and
3-ethyl-5-methyl-6-(2-pyridyl)-1,3-dihydrothiopheno[2,3-d]pyrimidine-2,4-dione.
   E. Preparation of a Compound of Formula I in Which R¹ is Ethyl, R² is Methyl, and R⁴ is Methyl

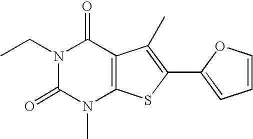

To a solution of 3-ethyl-6-(2-furyl)-5-methyl-1,3-dihydrothiopheno-[2,3-d]pyrimidine-2,4-dione (50 mg) in N,N-dimethylformamide (5 ml) was added potassium carbonate (100 mg) and methyl iodide (1 ml), and the mixture stirred at room temperature overnight. After removal of solvent under reduced pressure, the residue was chromatographed on silica gel, eluting with 20% ethyl acetate/hexane, to provide 3-ethyl-6-(2-furyl)-1,5-dimethyl-1,3-dihydrothiopheno[2,3-d]pyrimidine-2,4-dione.

¹H NMR (CDCl₃) δ 0.95 (m, 6H), 2.18 (m, 1H), 2.67 (s, 3H), 3.56 (s, 3H), 3.87 (d, 2H) 6.50 (m, 2H), 7.45 (d, 1H);

MS (EI): 318 (M⁺).

F. Preparation of Compounds of Formula I Varying R¹, R², and R³

Similarly, following the procedure of Example 4E above, but optionally substituting other compounds of Formula I for 3-ethyl-6-(2-furyl)-5-methyl-1,3-dihydrothiopheno-[2,3-d]pyrimidine-2,4-dione and other amides for N,N-dimethylformamide, the following compounds of Formula I were prepared:
3-ethyl-1,5-dimethyl-1,3-dihydrothiopheno[2,3-d]pyrimidine-2,4-dione;
3-ethyl-1,5-dimethyl-6-pyrrol-2-yl-1,3-dihydrothiopheno[2,3-d]pyrimidine-2,4-dione;
3-ethyl-6-(3-furyl)-1,5-dimethyl-1,3-dihydrothiopheno[2,3-d]pyrimidine-2,4-dione;
3-ethyl-1,5-dimethyl-6-(4-methyl(2-thienyl))-1,3-dihydrothiopheno[2,3-d]pyrimidine-2,4-dione;
3-ethyl-1,5-dimethyl-6-(3-methyl(2-thienyl))-1,3-dihydrothiopheno[2,3-d]pyrimidine-2,4-dione;
3-ethyl-1,5-dimethyl-6-(1,3-thiazol-2-yl)-1,3-dihydrothiopheno[2,3-d]pyrimidine-2,4-dione; and
3-ethyl-1,5-dimethyl-6-(2-pyridyl)-1,3-dihydrothiopheno[2,3-d]pyrimidine-2,4-dione.

### EXAMPLE 5

### Alternative Preparation of a Compound of Formula I

A. Preparation of a Compound of Formula I in Which R¹ is Ethyl and R² is Methyl

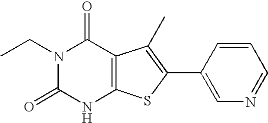

A mixture of 6-bromo-3-ethyl-5-methyl-1,3-dihydrothiopheno[2,3-d]pyrimidine-2,4-dione (24.4 mg 0.0844 mmol), 3-pyridineboronic acid (25.9 mg, 0.211 mmol), tetrakis(triphenylphosphine)palladium (4.9 mg, 0.00422 mmol), 2M aqueous sodium carbonate (0.5 ml) and N,N-dimethylformamide (2 ml) was introduced into a Smith Process Vial under argon. The sealed vial was heated using a microwave reactor at 160° C. for 10 minutes. The product was filtered through Celite (3 g), washing the celite with ethyl acetate (70 ml). The filtrate was washed with water (30 ml), followed by brine (30 ml) and dried over sodium sulfate. Solvent was removed from the dried solution under reduced pressure to give crude 3-ethyl-5-methyl-6-(3-pyridyl)-1,3-dihydrothiopheno[2,3-d]pyrimidine-2,4-dione as a brown oil. Crystallization from ethyl acetate (1 ml) gave the product as light brown crystals.

¹H NMR: (400 MHz, CDCl₃) 1;30 (3 H, t, J=7.0 Hz, —CH₂CH₃), 2.56 (3 H, s, CH₃(C5)), 4.10 (2H, q, J=7.0 Hz, —CH₂CH₃), 7.43 (1H, dd, J=8.6, 5.5 Hz, —CH(py-C5)), 7.79 (1H, dt, J=8.6, 2.0 Hz, —CH(py-C4)), 8.63 (1H, dd, J=5.5, 2.0 Hz, —CH(py-C6)), 8.71 (1H, d, J=2.0 Hz, —CH(py-C2)), 10.34 (1H, br s, NH);

MS: (EI): 287 (M⁺)

B. Preparation of a Compound of Formula I, varying R¹, R² and R³

Similarly, following the procedure of Example 5A above, but optionally substituting other compounds of formula (3) for 6-bromo-3-ethyl-5-methyl-1,3-dihydrothiopheno[2,3-d]pyrimidine-2,4-dione, and optionally substituting other boronic acid derivatives for 2-furanboronic acid, the following compounds of Formula I were prepared:
3-ethyl-5-methyl-6-pyrimidin-5-yl-1,3-dihydrothiopheno[2,3-d]pyrimidine-2,4-dione;
3-ethyl-5-methyl-6-(pyrrol-2-yl)-1,3-dihydrothiopheno[2,3-d]pyrimidine-2,4-dione;
3-ethyl-6-(6-methoxy(3-pyridyl))-5-methyl-1,3-dihydrothiopheno[2,3-d]pyrimidine-2,4-dione; and
3-ethyl-6-(2-methoxy(3-pyridyl))-5-methyl-1,3-dihydrothiopheno[2,3-d]pyrimidine-2,4-dione.

### EXAMPLE 6

### Preparation of a Compound of Formula (5)

A. Preparation of a Compound of Formula (5) in which R¹ is Ethyl and R² is Methyl

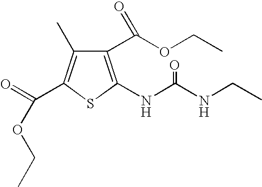

A mixture of propionic acid (0.8 ml, 10 mol), diphenylphosphoryl azide (2.22 ml, 10.3 mmol) and triethylamine (1.39 ml, 10 mmol) in toluene (15 ml) was refluxed for 90 minutes. The mixture was then cooled to below 40° C., and a solution of ethyl 2-amino-5-(ethoxycarbonyl)-4-methylthiophene-3-carboxylate (1.76 g, 6.85 mmol) in toluene (10 ml) was added, the mixture refluxed for 24 hours. The reaction mixture was diluted with brine, and extracted with ethyl acetate. The organic layer was washed with brine and dried over sodium sulfate. Solvent was removed under reduced pressure, and the residue purified by column chromatography, eluting with ethyl acetate/hexane 1:6 to 1:4, to provide ethyl 5-(ethoxycarbonyl)-2-[(ethylamino)carbonylamino]-4-methylthiophene-3-carboxylate. The structure was confirmed by ¹H NMR and mass spectrometry.

B. Preparation of other Compounds of Formula (5)

Similarly, following the procedure of Example 6A above, but optionally substituting ethyl 2-amino-5-(ethoxycarbonyl)-4-methylthiophene-3-carboxylate with other compounds of formula (4), and optionally substituting propionic acid with other carboxylic acids of formula R¹CO₂H, other compounds of formula (5) are prepared.

### EXAMPLE 7

### Preparation of a Compound of Formula (6)

A. Preparation of a Compound of Formula (6) in which R¹ is Ethyl and R² is Methyl

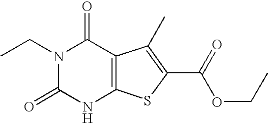

To a solution of ethyl 5-(ethoxycarbonyl)-2-[(ethylamino)carbonylamino]-4-methylthiophene-3-carboxylate (0.93 g, 2.84 mmol) in ethanol (20 ml) was added a solution of sodium ethoxide in ethanol (21% w/w, 2 ml). The mixture was stirred at room temperature overnight, cooled to 0° C., and acidified with 1N hydrochloric acid. Solvent was removed from the mixture under reduced pressure, and to the solid residue was added water, and the resulting solid filtered off, washed with water, and recrystallized from ethanol, to provide ethyl 3-ethyl-5-methyl-2,4-dioxo-1,3-dihydrothiopheno[2,3-d]pyrimidine-6-carboxylate. Mass spectrometry and ¹H NMR were satisfactory.

B. Preparation of other Compounds of Formula (6)

Similarly, following the procedure of Example 7A above, but replacing ethyl 5-(ethoxycarbonyl)-2-[(ethylamino)carbonylamino]-4-methylthiophene-3-carboxylate with other compounds of formula (5), other compounds of formula (6) are prepared:
ethyl 3-ethyl-5-methyl-2,4-dioxo-1,3-dihydrothiopheno[2,3-d]pyrimidine-6-carboxylate;
ethyl 3,5-dimethyl-2,4-dioxo-1,3-dihydrothiopheno[2,3-d]pyrimidine-6-carboxylate;
Ethyl 5-methyl-2,4-dioxo-3-prop-2-ynyl-1,3-dihydrothiopheno[2,3-d]pyrimidine-6-carboxylate; and
Ethyl 1,5-dimethyl-2,4-dioxo-3-prop-2-ynyl-1,3-dihydrothiopheno[2,3-d]pyrimidine-6-Carboxylate.

### EXAMPLE 8

### Preparation of a Compound of Formula (7)

A. Preparation of a Compound of Formula (7) in which R¹ is Ethyl and R² is Methyl

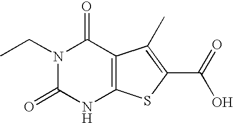

A solution of ethyl 3-ethyl-5-methyl-2,4-dioxo-1,3-dihydrothiopheno[2,3-d]pyrimidine-6-carboxylate (0.28 g, 1 mmol) and potassium hydroxide (0.28 g, 5 mmol) were dissolved in ethanol (20 ml) and stirred at 60° C. overnight. After cooling, solvent was removed form the mixture under reduced pressure, and the residue dissolved in water. The aqueous solution was acidifed with aqueous hydrochloric acid, and the precipitate thus produced filtered off, and washed with water, to provide 3-ethyl-5-methyl-2,4-dioxo-1,3-dihydrothiopheno[2,3-d]pyrimidine-6-carboxylic acid as a white solid, which was recrystallized from ethanol. Mass spectrometry and ¹H NMR were satisfactory.

B. Preparation of other Compounds of Formula (7)

Similarly, following the procedure of Example 8A above, but replacing ethyl 3-ethyl-5-methyl-2,4-dioxo-1,3-dihydrothiopheno[2,3-d]pyrimidine-6-carboxylate with other compounds of formula (6), other compounds of formula (7) were prepared:
5-methyl-2,4-dioxo-3-propyl-1,3-dihydrothiopheno[2,3-d]pyrimidine-6-carboxylic acid; and
5-methyl-2,4-dioxo-3-prop-2-ynyl-1,3-dihydrothiopheno[2,3-d]pyrimidine-6-carboxylic Acid.
   C. Preparation of other Compounds of Formula (7)

Similarly, following the procedure of Example 8A above, but replacing ethyl 3-ethyl-5-methyl-2,4-dioxo-1,3-dihydrothiopheno[2,3-d]pyrimidine-6-carboxylate with other compounds of formula (6), other compounds of formula (7) are prepared.

### EXAMPLE 9

### Preparation of a Compound of Formula (8)

A. Preparation of a Compound of Formula (8) in which R¹ is Ethyl and R² is Methyl

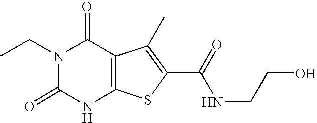

To a solution of 3-ethyl-5-methyl-2,4-dioxo-1,3-dihydrothiopheno[2,3-d]pyrimidine-6-carboxylic acid (92 mg, 0.36 mmol) and 2-aminoethan-1-ol (33 mg, 0.54 mmol) in methylene chloride 10 ml) at 0° C. was added 1-(3-dimethylaminopropyl)-3-ethylcarbodiimide hydrochloride (0.1 g, 0.54 mmol), 1-hydroxybenzotriazole (73 mg, 0.54 mmol), and triethylamine (55 mg, 0.54 mmol). The mixture was stirred at room temperature overnight, diluted with methylene chloride, and washed with 0.5 N hydrochloric acid. Some product was still present in the aqueous phase, and was extracted with methylene chloride. The organic layers were combined, dried over sodium sulfate, and solvent removed under reduced pressure. Methanol and methylene chloride were added to the residue, and the white solid filtered off and washed with methylene chloride, to provide (3-ethyl-5-methyl-2,4-dioxo(1,3-dihydrothiopheno[2,3-d]pyrimidin-6-yl))-N-(2-hydroxyethyl)carboxamide. Mass spectrometry and ¹H NMR were satisfactory.

B. Preparation of other Compounds of Formula (8)

Similarly, following the procedure of Example 9A above, but replacing 3-ethyl-5-methyl-2,4-dioxo-1,3-dihydrothiopheno[2,3-d]pyrimidine-6-carboxylic acid with other compounds of formula (7), other compounds of formula (8) were prepared:
N-((1R)-2-hydroxy-isopropyl)(3-ethyl-5-methyl-2,4-dioxo(1,3-dihydrothiopheno[2,3-d]pyrimidin-6-yl))carboxamide;
N-((1S)-2-hydroxy-isopropyl)(3-ethyl-5-methyl-2,4-dioxo(1,3-dihydrothiopheno[2,3-d]pyrimidin-6-yl))carboxamide;
N-((1R)-2-hydroxy-isopropyl)(5-methyl-2,4-dioxo-3-propyl(1,3-dihydrothiopheno[2,3-d]pyrimidin-6-yl))carboxamide;
N-((1S)-2-hydroxy-isopropyl)(5-methyl-2,4-dioxo-3-propyl(1,3-dihydrothiopheno[2,3-d]pyrimidin-6-yl))carboxamide;
(3-ethyl-5-methyl-2,4-dioxo(1,3-dihydrothiopheno[2,3-d]pyrimidin-6-yl))-N,N-dimethylcarboxamide; and
6-[(4S)-4-cyclohexylmethyl)(1,3-oxazolin-2-yl)]-5-methyl-3-(2-methylpropyl)-1,3-dihydrothiopheno[2,3-d]pyrimidine-2,4-dione.
   C. Preparation of other Compounds of Formula (8)

Similarly, following the procedure of Example 9A above, but replacing 3-ethyl-5-methyl-2,4-dioxo-1,3-dihydrothiopheno[2,3-d]pyrimidine-6-carboxylic acid with other compounds of formula (7), other compounds of formula (8) are prepared.

### EXAMPLE 10

### Preparation of a Compound of Formula I

A. Preparation of a Compound of Formula I in which R¹ is Ethyl, R² is Methyl, and R³ is 1,3-Oxazolin-2-yl.

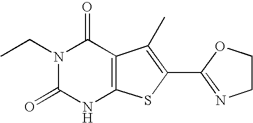

A mixture of (3-ethyl-5-methyl-2,4-dioxo(1,3-dihydrothiopheno[2,3-d]pyrimidin-6-yl))-N-(2-hydroxyethyl)carboxamide (70 mg, 0.24 mmol), Burgess reagent ((methoxycarbonylsulfamoyl)triethylammonium hydroxide inner salt, 64 mg, 0.27 mmol) in tetrahydrofuran (4 ml) was stirred at 70° C. for 2 hours. Solvent was removed from the mixture under reduced pressure, and the residue purified by preparative thin layer chromatography, eluting with 5% methanol/methylene chloride, to provide 3-ethyl-5-methyl-6-(1,3-oxazolin-2-yl)-1,3-dihydrothiopheno[2,3-d]pyrimidine-2,4-dione as a white solid. Mass spectrometry and ¹H NMR were satisfactory.

¹H-NMR (CD3OD) 1.23 (t, J=7.04 Hz, 3H), 2.79 (s, 3H), 3.95-4.05 (m, 4H), 4.50 (t, 2H);

MS (EI): 279 (M⁺))

B. Preparation of other Compounds of Formula I with varying R³.

Similarly, following the procedure of Example 10A above, but replacing (3-ethyl-5-methyl-2,4-dioxo(1,3-dihydrothiopheno[2,3-d]pyrimidin-6-yl))-N-(2-hydroxyethyl)carboxamide with other compounds of formula (8), the following other compounds of Formula I were prepared:
5-methyl-3-(2-methylpropyl)-6-(1,3-oxazolin-2-yl)-1,3-dihydrothiopheno[2,3-d]pyrimidine-2,4-dione
6-((4R)-4-methyl(1,3-oxazolin-2-yl))-3-ethyl-5-methyl-1,3-dihydrothiopheno[2,3-d]pyrimidine-2,4-dione;
6-((4S)-4-methyl(1,3-oxazolin-2-yl))-3-ethyl-5-methyl-1,3-dihydrothiopheno[2,3-d]pyrimidine-2,4-dione;
6-((4R)-4-methyl(1,3-oxazolin-2-yl))-5-methyl-3-propyl-1,3-dihydrothiopheno[2,3-d]pyrimidine-2,4-dione;
6-((4S)-4-methyl(1,3-oxazolin-2-yl))-5-methyl-3-propyl-1,3-dihydrothiopheno[2,3-d]pyrimidine-2,4-dione;
6-((4R)-4-methyl(1,3-oxazolin-2-yl))-1,5-dimethyl-3-propyl-1,3-dihydrothiopheno[2,3-d]pyrimidine-2,4-dione;
6-((4S)-4-methyl(1,3-oxazolin-2-yl))-5-methyl-3-(2-methylpropyl)-1,3-dihydrothiopheno[2,3-d]pyrimidine-2,4-dione;
6-((4S)-4-methyl(1,3-oxazolin-2-yl))-1,5-dimethyl-3-(2-methylpropyl)-1,3-dihydrothiopheno[2,3-d]pyrimidine-2,4-dione;
6-((4S)-4-ethyl(1,3-oxazolin-2-yl))-5-methyl-3-(2-methylpropyl)-1,3-dihydrothiopheno[2,3-d]pyrimidine-2,4-dione;
6-((5R)-5-methyl(1,3-oxazolin-2-yl))-5-methyl-3-(2-methylpropyl)-1,3-dihydrothiopheno[2,3-d]pyrimidine-2,4-dione;
6-((4S)-4-ethyl(1,3-oxazolin-2-yl))-1,5-dimethyl-3-(2-methylpropyl)-1,3-dihydrothiopheno[2,3-d]pyrimidine-2,4-dione; and
6-((5R)-5-methyl(1,3-oxazolin-2-yl))-1,5-dimethyl-3-(2-methylpropyl)-1,3-dihydrothiopheno[2,3-d]pyrimidine-2,4-dione.
   C. Preparation of other Compounds of Formula I in which R³ is 1,3-Oxazolin-2-yl.

Similarly, following the procedure of Example 10A above, but replacing (3-ethyl-5-methyl-2,4-dioxo(1,3-dihydrothiopheno[2,3-d]pyrimidin-6-yl))-N-(2-hydroxyethyl)carboxamide with other compounds of formula (8), other compounds of Formula I are prepared.

### EXAMPLE 11

### Preparation of a Compound of Formula I

A. Preparation of a Compound of Formula I in which R¹ is Ethyl, R² is Methyl, and R³ is Oxazol-2-yl.

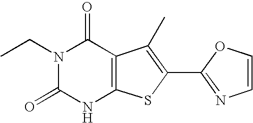

A solution of 3-ethyl-5-methyl-6-(1,3-oxazolin-2-yl)-1,3-dihydrothiopheno[2,3-d]pyrimidine-2,4-dione (10 mg, 0.036 mmol) in toluene was heated to 50° C., and 2,3-dichloro-5,6-dicyano-1,4-benzoquinone (41 mg, 0.18 mmol) added in small portions. The resulting solution was stirred at 95° C. overnight, colled, and the solvent removed under reduced pressure. The residue was purified by preparative thin layer chromatography, eluting with 5% methanol/methylene chloride, to provide 3-ethyl-5-methyl-6-(1,3-oxazol-2-yl)-1,3-dihydrothiopheno[2,3-d]pyrimidine-2,4-dione as a light yellow solid.

¹H-NMR (CD3OD) 1.23 (t, J=7.04 Hz, 3H), 2.84 (s, 3H), 4.02 (q, 2H), 7.26 (s, 1H), 7.96 (s, 1H);

MS (EI): 277 (M⁺)

B. Preparation of other Compounds of Formula I.

Similarly, following the procedure of Example 11A above, but replacing 3-ethyl-5-methyl-6-(1,3-oxazolin-2-yl)-1,3-dihydrothiopheno[2,3-d]pyrimidine-2,4-dione with other compounds of Formula I wherein R³ is an unsaturated heterocycle, other compounds of Formula I were prepared:
5-methyl-3-(2-methylpropyl)-6-(1,3-oxazol-2-yl)-1,3-dihydrothiopheno[2,3-d]pyrimidine-2,4-dione
1,5-dimethyl-3-(2-methylpropyl)-6-(1,3-oxazol-2-yl)-1,3-dihydrothiopheno[2,3-d]pyrimidine-2,4-dione;and
3-ethyl-1,5-dimethyl-6-(1,3-thiazol-2-yl)-1,3-dihydrothiopheno[2,3-d]pyrimidine-2,4-dione.
   C. Preparation of other Compounds of Formula I in which R³ is 1,3-oxazol-2-yl

Similarly, following the procedure of Example 11A above, but replacing 3-ethyl-5-methyl-6-(1,3-oxazolin-2-yl)-1,3-dihydrothiopheno[2,3-d]pyrimidine-2,4-dione with other compounds of Formula I in which R³ is 1,3-oxazolin-2-yl, other compounds of Formula I are prepared in which R³ is 1,3-oxazol-2-yl.

### EXAMPLE 12

### Preparation of a Compound of Formula (9)

A. Preparation of a Compound of Formula (9) in which R¹ is Ethyl and R² is Methyl

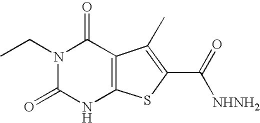

A suspension of ethyl 3-ethyl-5-methyl-2,4-dioxo-1,3-dihydrothiopheno[2,3-d]pyrimidine-6-carboxylate (1 g, 3.3 mmol) in hydrazine monohydrate (7 ml) was stirred at 120° C. for 3 hours. The liquid was removed under reduced pressure, and the residue triturated with diethyl ether (10 ml), filtered, washed with ether, and dried under reduced pressure, to provide 3-ethyl-5-methyl-1,3-dihydrothiopheno[2,3-d]pyrimidine-2,4-dione-6-carbohydrazide, which was used in the next step with no further purification.

B. Preparation of other Compounds of Formula (9)

Similarly, following the procedure of Example 12A above, but replacing ethyl 3-ethyl-5-methyl-2,4-dioxo-1,3-dihydrothiopheno[2,3-d]pyrimidine-6-carboxylate by other compounds of formula (6), other compounds of formula (9)are prepared.

### EXAMPLE 13

### Preparation of a Compound of Formula I

A. Preparation of a Compound of Formula I in which R¹ is Ethyl, R² is Methyl, and R³ is 1,3,4-Oxadiazolyl

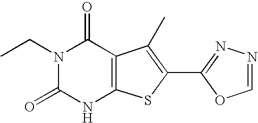

A suspension of 3-ethyl-5-methyl-1,3-dihydrothiopheno[2,3-d]pyrimidine-2,4-dione-6-carbohydrazide (50 mg, 0.2 mmol) in a mixture of acetic acid (2 ml) and triethylorthoformate (2 ml) was heated in a microwave oven at 160° C. for 20 minutes. After standing at room temperature a precipitate formed, which was filtered off, washed with acetic acid, and dried under reduced pressure to provide 3-ethyl-5-methyl-6-(1,3,4-oxadiazol-2-yl)-1,3-dihydrothiopheno[2,3-d]pyrimidine-2,4-dione.

B. Preparation of other Compounds of Formula I in which R³ is 1,3,4-Oxadiazol-2-yl.

Similarly, following the procedure of Example 13A above, but replacing triethylorthoformate with other compounds of formula RC(OEt)₃ in which R is methyl, propyl, or phenyl, the following compounds of Formula I were prepared:
3-ethyl-5-methyl-6-(5-methyl(1,3,4-oxadiazol-2-yl))-1,3-dihydrothiopheno[2,3-d]pyrimidine-2,4-dione;
3-ethyl-6-(5-ethyl(1,3,4-oxadiazol-2-yl))-5-methyl-1,3-dihydrothiopheno[2,3-d]pyrimidine-2,4-dione; and
3-ethyl-5-methyl-6-(5-phenyl(1,3,4-oxadiazol-2-yl))-1,3-dihydrothiopheno[2,3-d]pyrimidine-2,4-dione.
   C. Preparation of other Compounds of Formula I in which R³ is 1,3,4-Oxadiazol-2-yl

Similarly, following the procedure of Example 13A above, but optionally replacing 3-ethyl-5-methyl-1,3-dihydrothiopheno[2,3-d]pyrimidine-2,4-dione-6-carbohydrazide with other compounds of formula (9), and optionally replacing triethylorthoformate with other compounds of formula RC(OEt)₃, other compounds of Formula I in which R³ is a 1,3,4-oxadiazol-2-yl derivative are prepared.

### EXAMPLE 14

### Preparation of a Compound of Formula I in which R³ is an Amide

A. Preparation of a Compound of Formula I in which R¹ is Ethyl, R² is Methyl, and R³ is Methylaminocarbonyl

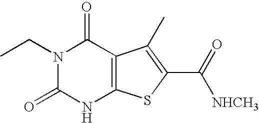

A solution of ethyl 3-ethyl-5-methyl-2,4-dioxo-1,3-dihydrothiopheno[2,3-d]pyrimidine-6-carboxylate (30 mg) in methanol (2 ml) was stirred with 40% methylamine in water (5 ml) at 60° C. in a stirred tube overnight. Solvent was removed from the reaction mixture under reduced pressure, and the residue was purified by preparative thin layer chromatography, eluting with 5% methanol/methylene chloride, to provide (3-ethyl-5-methyl-2,4-dioxo(1,3-dihydrothiopheno[2,3-d]pyrimidin-6-yl))-N-methylcarboxamide as a white solid.

B. Preparation of other Compounds of Formula I in which R³ is an Amide

Similarly, following the procedure of Example 14A above, but replacing ethyl 3-ethyl-5-methyl-2,4-dioxo-1,3-dihydrothiopheno[2,3-d]pyrimidine-6-carboxylate with other compounds of formula (6), other compounds of Formula I in which R³ is an amide are prepared:
(3,5-dimethyl-2,4-dioxo(1,3-dihydrothiopheno[2,3-d]pyrimidin-6-yl))-N-methylcarboxamide; and
N-methyl(5-methyl-2,4-dioxo-3-propyl(1,3-dihydrothiopheno[2,3-d]pyrimidin-6-yl))carboxamide.

### EXAMPLE 15

### Preparation of a Compound of Formula (10)

A. Preparation of a Compound of Formula (10) in which R¹ is Ethyl and R² is Methyl

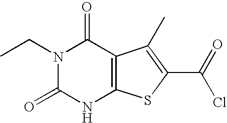

To a solution of 3-ethyl-5-methyl-2,4-dioxo-1,3-dihydrothiopheno[2,3-d]pyrimidine-6-carboxylic acid (0.13 g, 0.5 mmol) in tetrahydrofuran at ° C. was added oxalyl chloride (0.87 ml, 10 mmol) dropwise, and the mixture stirred overnight. Solvent was then removed under reduced pressure, to provide 3-ethyl-5-methyl-2,4-dioxo-1,3-dihydrothiopheno[2,3-d]pyrimidine-6-carbonyl chloride as a yellow oil, which was used in the next reaction with no further purification.

B. Preparation of other Compounds of Formula (10)

Similarly, following the procedure of Example 15A above, but replacing 3-ethyl-5-methyl-2,4-dioxo-1,3-dihydrothiopheno[2,3-d]pyrimidine-6-carboxylic acid with other compounds of formula (7), other compounds of formula (10) are prepared.

### EXAMPLE 16

### Preparation of a Compound of Formula (11)

A. Preparation of a Compound of Formula (11) in which R¹ is Ethyl and R² is Methyl

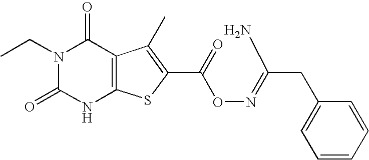

To a solution of N-hydroxy-2-phenylethanimidamide (75 mg, 0.5 mmol) in tetrahydrofuran at 0° C. was added diisopropylethylamine (0.17 ml, 1 mmol), followed by 3-ethyl-5-methyl-2,4-dioxo-1,3-dihydrothiopheno[2,3-d]pyrimidine-6-carbonyl chloride (136 mg, 0.5 mmol) dissolved in 4 ml of tetrahydrofuran. The temperature was allowed to warm to room temperature, and the mixture stirred for 24 hours. Solvent was removed under reduced pressure, and the residue purified by thin layer chromatography, eluting with 5% methanol/methylene chloride, to provide 2-amino-3-phenyl-1-azaprop-1-enyl-3-ethyl-5-methyl-2,4-dioxo-1,3-dihydrothiopheno[2,3-d]pyrimidine-6-carboxylate as a light yellow solid.

B. Preparation of other Compounds of Formula (11) in which R³ is 1,3,4-Oxadiazol-2-yl.

Similarly, following the procedure of Example 16A above, but replacing 3-ethyl-5-methyl-2,4-dioxo-1,3-dihydrothiopheno[2,3-d]pyrimidine-6-carbonyl chloride with other compounds of formula (10), other compounds of formula (11) are prepared.

### EXAMPLE 17

### Preparation of a Compound of Formula I

A. Preparation of a Compound of Formula I in which R¹ is Ethyl and R² is Methyl

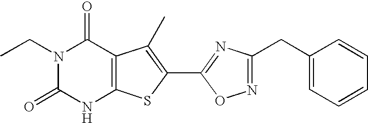

A mixture of ethyl 3-ethyl-5-methyl-2,4-dioxo-1,3-dihydrothiopheno[2,3-d]pyrimidine-6-carboxylate (50 mg, 0.13 mmol) and toluene (15 ml) was refluxed for 60 hours. Solvent was removed from the reaction mixture under reduced pressure, and the residue was purified by preparative thin layer chromatography, eluting with 5% methanol/methylene chloride, to provide crude 3-ethyl-5-methyl-6-[3-benzyl(1,2,4-oxadiazol-5-yl)]-1,3-dihydrothiopheno[2,3-d]pyrimidine-2,4-dione, which was treated with methanol and filtered, to produce a light yellow solid.

¹H-NMR (DMSO) 1.12 (t, J=7.04 Hz, 3H), 2.81 (s, 3H), 3.87 (q, 2H), 4.13 (s, 2H), 7.25-7.37 (m, 5H), 12.53 (s, 1H);

MS (EI): 368 (M⁺)

B. Preparation of other Compounds of Formula I

Similarly, following the procedure of Example 17A above, but replacing ethyl 3-ethyl-5-methyl-2,4-dioxo-1,3-dihydrothiopheno[2,3-d]pyrimidine-6-carboxylate with other compounds of formula (11), other compounds of Formula I in which R³ is a substituted oxadiazole are prepared.

### EXAMPLE 18

### Preparation of a Compound of Formula I

A. Preparation of a compound of Formula (2) in which R¹ is 2-methylpropyl, R² is methyl, and R³ is acetyl

Similarly, following the procedure of Example 1A above, but optionally substituting other compounds of formula (1) for ethyl 2-amino-4-methylthiophene-3-carboxylate, and optionally substituting other carboxylic acids of formula R¹CO₂H for acetic acid, the following compound of formula (2) was prepared:
ethyl 5-acetyl-4-methyl-2-{[(2-methylpropyl)amino]carbonylamino}thiophene-3-carboxylate.
   B. Preparation of a Compound of Formula I in which R¹ is 2-methylpropyl, R² is methyl, and R³ is acetyl

Similarly, following the procedure of Example 2A above, but substituting other compounds of formula (2) for ethyl 4-methyl-2-[(methylamino)carbonylamino]-thiophene-3-carboxylate, the following compound of formula I was prepared:
6-acetyl-5-methyl-3-(3-methylpropyl)-1,3-dihydrothiopheno[2,3-d]pyrimidine-2,4-dione.
   C. Preparation of a Compound of the formula in which R¹ is 2-methylpropyl, R² is methyl, and R³ is 2-bromoacetyl

To a stirred suspension of the compound of step B above (0.28 g., 1 mmol) in CH₂Cl₂ (10 mL) was added bromine (0.056 mL, 1.1 mmol) in CH₂Cl₂ (5 mL) dropwise in an ice bath. The resulting mixture was refluxed for 1 h. To the cooled reaction mixture was added saturated NaHCO₃ aqueous solution. The organic layer was separated and further washed with NaHCO₃ aqueous solution, then dried over Na₂SO₄, evaporated in vacuo to provide 6-(2-bromoacetyl)-5-methyl-3-(2-methylpropyl)-1,3-dihydrothiopheno[2,3-d]pyrimidine-2,4-dione. The residue was used directly for the next step.

D. Preparation of a Compound of Formula I in which R¹ is 2-methylpropyl, R² is methyl and R⁴ is hydrogen

The compound from step C above (0.34g, 1 mmol) was stirred in formamide (5 mL) at 180° C. for 1 h. The reaction mixture was then diluted with ethyl acetate and washed with water. The organic layer was dried over Na₂SO₄ and evaporated in vacuo. The crude product was purified by prep-TLC (EtOAc: Hexanes=2:3) to provide 5-methyl-3-(2-methylpropyl)-6-(1,3-oxazol-4-yl)-1,3-dihydrothiopheno[2,3-d]pyrimidine-2,4-dione (0.04 g).

E. Preparation of a Compound of Formula I in which R¹ is 2-methylpropyl, R² is methyl, R⁴ is hydrogen, varying R³

Similarly, following the procedure of step E above, but substituting other compounds for formamide, the following compound of formula I was prepared:
5-methyl-6-(2-methyl(1,3-thiazol-4-yl))-3-(2-methylpropyl)-1,3-dihydrothiopheno[2,3-d]pyrimidine-2,4-dione.

### EXAMPLE 19

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### EXAMPLE 20

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### EXAMPLE 21

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### EXAMPLE 22

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### EXAMPLE 23

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### EXAMPLE 24

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### EXAMPLE 25

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### EXAMPLE 26

Suspensions, each containing 50 mg of active ingredient per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### EXAMPLE 27

A subcutaneous formulation may be prepared as follows:

| Ingredient | Quantity | |
|---|---|---|
| Active Ingredient | 5.0 | mg |
| Corn Oil | 1.0 | mL |

### EXAMPLE 28

An injectable preparation is prepared having the following composition:

| Ingredients | Amount | |
|---|---|---|
| Active ingredient | 2.0 | mg/mL |
| Mannitol, USP | 50 | mg/mL |
| Gluconic acid, USP | q.s. (pH 5-6) | |
| water (distilled, sterile) | q.s. to 1.0 | mL |

| | |
|---|---|
| Nitrogen Gas, NF | q.s. |

### EXAMPLE 29

A topical preparation is prepared having the following composition:

| Ingredients | grams |
|---|---|
| Active ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### EXAMPLE 30

Sustained Release Composition

| | Weight | Preferred | Most |
|---|---|---|---|
| Ingredient | Range (%) | Range (%) | Preferred |
| Active ingredient | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release formulations of this invention are prepared as follows: compound and pH-dependent binder and any optional excipients are intimately mixed(dry-blended). The dry-blended mixture is then granulated in the presence of an aqueous solution of a strong base that is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, for example sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl, methylcellulose, cationic methacrylate copolymers (dimethylaminoethyl methacrylate/methyl-butyl methacrylate copolymers—Eudragit® E—Röhm. Pharrna), and the like. These film-forming agents may optionally contain colorants, plasticizers, and other supplemental ingredients.

The compressed tablets for example have a hardness sufficient to withstand 8 Kp compression. The tablet size will depend primarily upon the amount of compound in the tablet. The tablets will include from 300 to 1100 mg of compound free base. For example, the tablets will include amounts of compound free base ranging from 400-600 mg, 650-850 mg, and 900-1100 mg.

In order to influence the dissolution rate, the time during which the compound containing powder is wet mixed is controlled. For example the total powder mix time, i.e. the time during which the powder is exposed to sodium hydroxide solution, will range from 1 to 10 minutes and for example from 2 to 5 minutes. Following granulation, the particles are removed from the granulator and placed in a fluid bed dryer for drying at about 60° C.

### EXAMPLE 31

### A_{2A} Binding Assays

### Determination of A_{2A} Antagonist Activity

Reagents:

A tritiated adenosine A_{2A} antagonist, 3-(3-hydroxypropyl)-7-methyl-8-(m-methoxystyryl)-1-propargylxanthine, (3H-MSX-2, specific activity: 80 Ci/mmol), was purchased from American Radiolabeled Chemicals, Inc (St. Louis, Mo.). A tritiated adenosine A₁ antagonist, 1,3-Dipropyl-8-cyclopentylxanthine ([³H]DPCPX, specific activity: 120 Ci/mmol) was purchased from Perkin Elmer (Boston, Mass.). A tritiated adenosine A₂ₐ antagonist, 4-(2-[7-amino-2-(2-furyl)[1,2,4]triazolo[2,3-a][1,3,5]triazin-5-ylamino]ethyl)phenol ([³H]ZM241385 specific activity: 27.4 Ci/mmol) was purchased from American Radiolabeled Chemicals, Inc (St. Louis, Mo.). Adenosine Deaminase (ADA) was purchased from Roche Molecular Biochemicals (Nutley, N.J.). GTP was purchased from Sigma. Concentrated stock solution (10 mM) of compounds was dissolved in dimethylsulfoxide (DMSO), stored at −20° C., and diluted in Tris-EDTA buffer (50 mM Tris and 1 mM EDTA, 10 mM MgCl₂, pH 7.4) for use in experiments. The final content of dimethylsulfoxide in Tris-EDTA buffer during experiments was not more than 1%. Male Sprague Dawley rats, weighted 250-400 g, 8-10 weeks old, were purchased from Charles River Labs (Wilmington, Mass.).

Cell Culture

HEK293 (Human Embryonic Kidney 293) cells stably expressing human A_{2A} adenosine receptor or human A_{2B} adenosine receptor were maintained in DMEM supplemented with 10% fetal bovine serum, 2 mM L-glutamine, 100 IU/ml penicillin, 50 μg/ml streptomycin and 2 μg/ml puromycin. CHO (Chinese Hamster Ovary) cells stably expressing human A₁ adenosine receptor or human A₃ adenosine receptor were maintained in F12K medium supplemented with 10% fetal bovine serum, 2 mM L-glutamine, 100 IU penicillin, 50 μg streptomycin and 8 μg/ml puromycin. PC12 (rat pheochromocytoma) cells were cultured in F12K medium supplemented with 10% horse serum and 2.5% fetal bovine serum, 2 mM L-glutamine, 100 IU/ml penicillin, and 50 μg/ml streptomycin. All cells were maintained at 37° C. in a humidified 5% CO₂/95% air incubator and recultivated 2 times per week.

Membrane Preparations

Cultured cells in 150 mm² dishes were washed once with PBS, detached by scrapping, collected with buffer A (10 mM HEPES, 10 mM EDTA, pH 7.4) containing protease inhibitor cocktail. Cells were then homogenized by a handheld homogenizer at a speed of 4.5 for 1 min. The homogenate was centrifuged by a Beckman ultracentrifuge at a speed of 29,000 × g for 15 min. The pellets were resuspended in buffer HE (10 mM HEPES, 1 mM EDTA, pH 7.4, with protease inhibitor cocktail), centrifuged again at 29,000 × g for 15 min. The crude membrane was resuspended using buffer HE, and protein concentration was measured by the method of Lowery with BSA as standard. Similar procedures were used for membrane preparation for fresh rat tissues. All experimental procedures were done at 4° C. Membranes were aliquot and kept at −80° C.

Radioligand Binding Assays:

The binding assays utilized 15 ug of A_{2A} membrane (human recombinant A_{2A} membrane, rat striatal membrane, or PC 12 cell membrane) that had been treated with adenosine deaminase and 50 mM Tris-EDTA buffer (pH=7.4) followed by mixing 2 μL of serially diluted DMSO stock solution of the compounds of this invention at concentrations ranging from 10 μM to 0.1 nM or the control received 2 μL of DMSO alone, then the tritiated antagonist 3-(3-hydroxypropyl)-7-methyl-8-(m-methoxystyryl)-1-propargylxanthine (³H-MSX-2) in Tris-EDTA buffer (50 mM Tris, 1 mM EDTA, 10 mM MgCl₂, pH of 7.4) was added to achieve a final concentration of 2 nM. After incubation at 23° C. for 2 hours, the solutions were filtered using a membrane harvester with multiple washing of the membranes (3×). The filter plates were counted in scintillation cocktail affording the amount of displacement ³H-MSX-2 by the competitive binding compositions of this invention. Radioligand binding data was analyzed using GraphPad Prism version 4.0 (San Diego, Calif.). When appropriate, the significance of differences among 3 or more individual mean values was determined by one-way ANOVA followed by Student-Newman-Keuls test. A P value less than 0.05 was considered to indicate a statistically significant difference.

Using the above assays, Ki(A_{2A}) data was generated for the compounds of the invention. Data for a number of representative compounds is presented in Table 1 below.

| EXAMPLE | | |
|---|---|---|
| No. | NAME | K_{I}(A_{2A}) NM |
| I. | 6-(2-furyl)-5-methyl-3-(2-methylpropyl)-1,3- | 76 |
| | dihydrothiopheno[2,3-d]pyrimidine-2,4-dione | |
| II. | 3-ethyl-6-(3-furyl)-5-methyl-1,3-dihydrothiopheno[2,3- | 88 |
| | d]pyrimidine-2,4-dione | |
| III. | 3-ethyl-5-methyl-6-(2-thienyl)-1,3-dihydrothiopheno[2,3- | 97 |
| | d]pyrimidine-2,4-dione | |
| IV. | 3-ethyl-6-(2-furyl)-5-methyl-1,3-dihydrothiopheno[2,3- | 105 |
| | d]pyrimidine-2,4-dione | |
| V. | 3-ethyl-5-methyl-6-(1,3-oxazol-2-yl)-1,3- | 134 |
| | dihydrothiopheno[2,3-d]pyrimidine-2,4-dione | |
| VI. | 3-ethyl-5-(2-furyl)-1,3-dihydrothiopheno[2,3- | 137 |
| | d]pyrimidine-2,4-dione | |
| VII. | 3-ethyl-5-methyl-6-(1,3-oxazolin-2-yl)-1,3- | 152 |
| | dihydrothiopheno[2,3-d]pyrimidine-2,4-dione | |
| VIII. | (3,5-dimethyl-2,4-dioxo(1,3-dihydrothiopheno[2,3- | 274 |
| | d]pyrimidin-6-yl))-n-methylcarboxamide | |
| IX. | 5-methyl-3-prop-2-ynyl-1,3-dihydrothiopheno[2,3- | 365 |
| | d]pyrimidine-2,4-dione | |
| X. | 3-ethyl-5-methyl-1,3-dihydrothiopheno[2,3-d]pyrimidine- | 397 |
| | 2,4-dione | |
| XI. | 3-ethyl-1,3,5,6,7,8-hexahydrobenzo[b]thiopheno[2,3- | 458 |
| | d]pyrimidine-2,4-dione | |
| XII. | 3-ethyl-5-methyl-6-(3-thienyl)-1,3-dihydrothiopheno[2,3- | 559 |
| | d]pyrimidine-2,4-dione | |
| XIII. | 3,5-dimethyl-1,3-dihydrothiopheno[2,3-d]pyrimidine-2,4- | 648 |
| | dione | |
| XIV. | n-methyl(5-methyl-2,4-dioxo-3-propyl(1,3- | 650 |
| | dihydrothiopheno[2,3-d]pyrimidin-6-yl))carboxamide | |
| XV. | (3-ethyl-5-methyl-2,4-dioxo(1,3-dihydrothiopheno[2,3- | 676 |
| | d]pyrimidin-6-yl))-n-methylcarboxamide | |
| XVI. | 3-ethyl-5,6-dimethyl-1,3-dihydrothiopheno[2,3- | 699 |
| | d]pyrimidine-2,4-dione | |
| XVII. | ethyl 3,5-dimethyl-2,4-dioxo-1,3-dihydrothiopheno[2,3- | 715 |
| | d]pyrimidine-6-carboxylate | |
| XVIII. | 3-ethyl-5-methyl-6-[1-benzylpyrazol-4-yl]-1,3- | 752 |
| | dihydrothiopheno[2,3-d]pyrimidine-2,4-dione | |
| XIX. | 5-methyl-3-(2-methylpropyl)-1,3-dihydrothiopheno[2,3- | 780 |
| | d]pyrimidine-2,4-dione | |
| XX. | 3-ethyl-5-methyl-6-[3-benzyl(1,2,4-oxadiazol-5-yl)]-1,3- | 805 |
| | dihydrothiopheno[2,3-d]pyrimidine-2,4-dione | |
| XXI. | 3-ethyl-5-(5-methyl(2-furyl))-1,3-dihydrothiopheno[2,3- | 230 |
| | d]pyrimidine-2,4-dione | |
| XXII. | 3-ethyl-1-methyl-5-(5-methyl(2-furyl))-1,3- | 896 |
| | dihydrothiopheno[2,3-d]pyrimidine-2,4-dione | |
| XXIII. | 5-methyl-3-(2-methylpropyl)-6-(1,3-oxazol-4-yl)-1,3- | 96 |
| | dihydrothiopheno[2,3-d]pyrimidine-2,4-dione | |
| XXIV. | 1,5-dimethyl-3-(2-propylmethyl)-6-(1,3-oxazol-4-yl)-1,3- | 165 |
| | dihydrothiopheno[1,3-d]pyrimidine-2,4-dione | |
| XXV. | ethyl 5-methyl-2,4-dioxo-3-prop-2-ynyl-1,3- | 300 |
| | dihydrothiopheno[2,3-d]pyrimidine-6-carboxylate | |
| XXVI. | ethyl 1,5-dimethyl-2,4-dioxo-3-prop-2-ynyl-1,3- | 49 |
| | dihydrothiopheno[2,3-d]pyrimidine-6-carboxylate | |
| XXVII. | 3-ethyl-5-methyl-6-pyrrol-2-yl-1,3-dihydrothiopheno[2,3- | 180 |
| | d]pyrimidine-2,4-dione | |
| XXVIII. | 5-methyl-3-(2-methylpropyl)-6-(1,3-oxazolin-2-yl)-1,3- | 137 |
| | dihydrothiopheno[2,3-d]pyrimidine-2,4-dione | |
| XXIX. | 5-methyl-3-(2-methylpropyl)-6-(1,3-oxazol-2-yl)-1,3- | 75 |
| | dihydrothiopheno[2,3-d]pyrimidine-2,4-dione | |
| XXX. | 6-(3-furyl)-5-methyl-3-(2-methylpropyl)-1,3- | 158 |
| | dihydrothiopheno[2,3-d]pyrimidine-2,4-dione | |
| XXXI. | 3-ethyl-5-methyl-6-(4-methyl(2-thienyl))-1,3- | 150 |
| | dihydrothiopheno[2,3-d]pyrimidine-2,4-dione | |
| XXXII. | 3-ethyl-5-methyl-6-(3-methyl(2-thienyl))-1,3- | 110 |
| | dihydrothiopheno[2,3-d]pyrimidine-2,4-dione | |
| XXXIII. | 3,5-dimethyl-6-(2-thienyl)-1,3-dihydrothiopheno[2,3- | 150 |
| | d]pyrimidine-2,4-dione | |
| XXXIV | 6-(2-furyl)-3,5-dimethyl-1,3-dihydrothiopheno[2,3- | 110 |
| | d]pyrimidine-2,4-dione | |
| XXXV | 3-(cyclopropylmethyl)-5-methyl-6-(2-thienyl)-1,3- | 195 |
| | dihydrothiopheno[2,3-d]pyrimidine-2,4-dione | |
| XXXVI | 3-(cyclopropylmethyl)-5-methyl-6-(2-furyl)-1,3- | 170 |
| | dihydrothiopheno[2,3-d]pyrimidine-2,4-dione | |
| XXXVII | 3-ethyl-5-methyl-6-(1,3-thiazol-2-yl)-1,3- | 167 |
| | dihydrothiopheno[2,3-d]pyrimidine-2,4-dione | |
| XXXVIII | 3-ethyl-5-methyl-6-(2-pyridyl)-1,3-dihydrothiopheno[2,3- | 120 |
| | d]pyrimidine-2,4-dione | |
| XXXIX | 3-ethyl-6-(2-furyl)-1,5-dimethyl-1,3- | 250 |
| | dihydrothiopheno[2,3-d]pyrimidine-2,4-dione | |
| XL | 1,5-dimethyl-3-(2-methylpropyl)-6-(1,3-oxazol-2-yl)-1,3- | 347 |
| | dihydrothiopheno[2,3-d]pyrimidine-2,4-dione | |
| XLI | 5-methyl-2,4-dioxo-3-propyl-1,3-dihydrothiopheno[2,3- | 569 |
| | d]pyrimidine-6-carboxylic acid | |
| XLII | N—((1R)-2-hydroxy-isopropyl)(3-ethyl-5-methyl-2,4- | 769 |
| | dioxo(1,3-dihydrothiopheno[2,3-d]pyrimidin-6- | |
| | yl))carboxamide | |
| XLIII | 6-((4S)-4-methyl(1,3-oxazolin-2-yl))-3-ethyl-5-methyl-1,3- | 93 |
| | dihydrothiopheno[2,3-d]pyrimidine-2,4-dione | |
| XLIV | 1-ethyl-6-(2-furyl)-5-methyl-3-(2-methylpropyl)-1,3- | 457 |
| | dihydrothiopheno[2,3-d]pyrimidine-2,4-dione | |
| XLV | 6-((4S)-4-methyl(1,3-oxazolin-2-yl))-5-methyl-3-propyl- | 95 |
| | 1,3-dihydrothiopheno[2,3-d]pyrimidine-2,4-dione | |
| XLVI | 3-ethyl-6-(3-furyl)-1,5-dimethyl-1,3- | 546 |
| | dihydrothiopheno[2,3-d]pyrimidine-2,4-dione | |
| XLVII | 3-ethyl-1,5-dimethyl-6-(4-methyl(2-thienyl))-1,3- | 524 |
| | dihydrothiopheno[2,3-d]pyrimidine-2,4-dione | |
| XLVIII | 3-ethyl-1,5-dimethyl-6-(1,3-thiazol-2-yl)-1,3- | 199 |
| | dihydrothiopheno[2,3-d]pyrimidine-2,4-dione | |
| XLIX | 3-ethyl-1,5-dimethyl-6-pyrrol-2-yl-1,3- | 488 |
| | dihydrothiopheno[2,3-d]pyrimidine-2,4-dione | |
| L | 3-ethyl-1,5-dimethyl-6-(2-pyridyl)-1,3- | 485 |
| | dihydrothiopheno[2,3-d]pyrimidine-2,4-dione | |
| LI | 6-((4S)-4-methyl(1,3-oxazolin-2-yl))-5-methyl-3-(2- | 48 |
| | methylpropyl)-1,3-dihydrothiopheno[2,3-d]pyrimidine-2,4- | |
| | dione | |
| LII | 1,5-dimethyl-3-prop-2-ynyl-1,3-dihydrothiopheno[2,3- | 720 |
| | d]pyrimidine-2,4-dione | |
| LIII | 6-((4S)-4-methyl(1,3-oxazolin-2-yl))-1,5-dimethyl-3-(2- | 304 |
| | methylpropyl)-1,3-dihydrothiopheno[2,3-d]pyrimidine-2,4- | |
| | dione | |
| LIV | 3-ethyl-6-(6-methoxy(3-pyridyl))-5-methyl-1,3- | 406 |
| | dihydrothiopheno[2,3-d]pyrimidine-2,4-dione | |
| LV | 3-ethyl-6-(2-methoxy(3-pyridyl))-5-methyl-1,3- | 529 |
| | dihydrothiopheno[2,3-d]pyrimidine-2,4-dione | |
| LVI | 6-((4S)-4-ethyl(1,3-oxazolin-2-yl))-5-methyl-3-(2- | 58 |
| | methylpropyl)-1,3-dihydrothiopheno[2,3-d]pyrimidine-2,4- | |
| | dione | |
| LVII | 6-((5R)-5-methyl(1,3-oxazolin-2-yl))-5-methyl-3-(2- | 239 |
| | methylpropyl)-1,3-dihydrothiopheno[2,3-d]pyrimidine-2,4- | |
| | dione | |
| LVIII | 6-((4S)-4-ethyl(1,3-oxazolin-2-yl))-1,5-dimethyl-3-(2- | 119 |
| | methylpropyl)-1,3-dihydrothiopheno[2,3-d]pyrimidine-2,4- | |
| | dione | |
| LIX | 6-((5R)-5-methyl(1,3-oxazolin-2-yl))-1,5-dimethyl-3-(2- | 450 |
| | methylpropyl)-1,3-dihydrothiopheno[2,3-d]pyrimidine-2,4- | |
| | dione | |

cAMP Assay

HEK293-A₂A cells and PC12 cells were seeded on 150×25 mm tissue culture dishes and grown 36-72 h to reach ˜80% confluence. Cells were then washed once with PBS and detached with PBS containing 2 mM EDTA. Cells were pelleted at a speed of 1000 rpm and resuspended in Opti-MEM I. ADA (1 U/ml) was added to eliminate adenosine. Cells were loaded into 96-well plates (˜6500 cells/well) and incubated with agonist in the absence or presence of antagonists (pretreated for 5 min) for 30 min at 37° C. The cAMP production was measured by using a DiscoveRx kit according to the manufacturer's instructions.

### EXAMPLE 32

Evaluation of Anti-Parkinsonian Activity in vivo Haloperidol-Induced Catalepsy in the Rat

This method assesses the ability of an animal to respond to an externally imposed posture after receiving the neuroleptic dopamine D2 antagonist haloperidol. Drugs which are effective in treating Parkinson's disease, such as L-DOPA, block haloperidol-induced catalepsy (Mandhane, S. N.; Chopde, C. T.; Ghosh, A. K. (1997). Adenosine A_{2A} receptors modulate haloperidol-induced catalepsy in rats.

The compounds of the invention are prepared in injectable form and diluted to a final concentration using physiological saline. 3,7-Dimethyl-1-propargylxanthine (DMPX) (0, 3 mg/kg) is dissolved in saline. All drugs are administered in a volume of 2 ml/kg. Animals receive three injections: (1) vehicle or compound p.o. 6 hours prior to testing, (2) haloperidol (0.2 mg/kg) i.p. 2.5 hours prior to testing, and (3) vehicle or DMPX (3 mg/kg) 30 minutes prior to testing.

The test procedure is as follows:

| | |
|---|---|
| Step I | The rat is taken out of the home cage and placed on a table. If |
| | the rat failed to move when touched gently on the back or |
| | pushed, a score of 0.5 is assigned. |
| Step II | The front paws of the rat are placed alternately on a 3 cm high |
| | wooden block. If the rat fails to correct this posture within 15 |
| | seconds, a score of 0.5 for each paw is added to the score of |
| | Step I. |
| Step III | The front paws of the rat are placed alternately on a 9 cm high |
| | wooden block. If the rat fails to correct the posture within 15 |
| | seconds, a score is added to the scores of Step I and II. Thus, for |
| | any animal, the highest score obtainable is 3.5 (cut-off score) |
| | reflecting total catalepsy. |

Data from the experiment are analysed using Kruskal-Wallis ANOVA followed by Mann-Whitney U test when appropriate, and are expressed as means +/− standard error of the mean *p < 0.05 versus vehicle control.

MPTP Lesion Model

Mice (C57/BL Harlan) receive a unilateral intrastriatal injection of the test compound, vehicle control, and positive control, in a volume of 1.0 .mu.l (15 mice per group). 30 min. after administration of the test compound all mice are systemically administered MPTP (N-methyl-4-phenyl-1,2,5,6-tetrahydropyridine) (25 mg/kg s.c), and this MPTP treatment is repeated 24 hours later. At suitable time points the spontaneous locomotor activity of the animals, as measured in automated activity monitors, is compared with control animals.

Animals are sacrificed 14 days after the second MPTP injection and striatal tissue is dissected out for HPLC analysis of dopamine and its metabolites, 3,4-dihydroxyphenylacetic acid and homovanillic acid. Reverse-phase UPLC in conjunction with electrochemical detection (Antec Decade detector, glossy carbon cell, set to +0.65 V versus a Ag/AgCl reference) is employed. The HPLC mobile phase consisted of 0.15 M NaH.sub.2 PO.sub.4, 0.1 mM EDTA, 0.55 mM octyl sulphate, 16% methanol (pH 3.6, adjusted with orthophosphoric acid).

The effects of test compounds on MPTP-induced mesencephalic damage is demonstrated by comparison with dopamine, 3,4-dihydroxyphenylacetic acid and homovanillic acid levels in caudate tissue taken ipsilateral and controlateral to the test compound injection. The influence of test compounds on MPTP-induced effects on locomotion and catecholamine and metabolite tissue levels is assessed by repeated measures analysis of variance (ANOVA) with appropriate tests.

### EXAMPLE 33

### Evaluation of Treatment of Alcohol Abuse in vivo in the Rat

Male Long Evans rats (Harlan, Indianapolis, Ind.), weighing approximately 250 g, are individually housed with food and water available ad libitum, and maintained on a 12 hour light/dark cycle. Ethanol dilution (10% v/v) for self-administration is prepared using 95% ethyl alcohol and tap water. Sucrose solution (10% w/v) is prepared using tap water. The test compound is dissolved in warm saline, and administered in a 1 ml/kg volume. Ethanol operant self-administration is carried out in standard operant chambers (Med Associates, Georgia, Vt.) housed in sound-attenuated cubicles. Each chamber (33×30.5×33 cm) contains two retractable levers against the right wall, 7 cm from the floor and 1 cm from the right or left edge of the right wall, respectively. One recessed dish positioned at 2.5 cm above floor level and 6 cm from the levers towards the center of the chamber is the reinforcer receptacle. Fluid (0.1 ml) is delivered from 10 syringe pumps upon activation of 1 of the 2 retractable response levers. A 3 second tone is activated upon lever pressing. Pressing the inactive lever resulted in no visual/auditory cue or reinforcement delivery, except during sucrose overnight sessions. The beginning of a training session is signaled by the onset of the house light located in the center of the wall facing the levers, at 27.2 cm above the floor. A computer-controlled stimulus and fluid delivery and recorded operant responses.

Before the beginning of the ethanol operant self-administration, rats are exposed to a 10% ethanol solution as the only liquid source in their home cages for 4 days. For the next 14 days, animals are allowed free choice between 10% ethanol solution in tap water or tap water from graded glass tubes. At the end of this 14-day period, operant self-administration is initiated according to the sucrose fading technique (Samson, 1986) with minor modifications. Rats are restricted to 30 minutes of water per day for 2 consecutive days. On the night of the second day of water restriction, rats are placed in the operant chambers for a 12-15 hours overnight session on an FR1 schedule (1 reinforcement of 0.1 ml per lever press) with 10%sucrose as a reinforcer and both levers active. The next day, rats begin the operant self-administration training. Animals are kept on water restriction for the next 4-5 days, during which they receive one 45 minute session per day on an FR1 schedule with 10% ethanol solution as a reinforcer and one active lever. They are then given free water in their home cages for the remainder of the experiment and are trained for 2-3 more of the above described sessions. The next day, sessions are shortened to 30 minutes and the ratio of responding was increased to an FR3 schedule. Ethanol is added to the sweet solution (10% sucrose/10% ethanol), and rats receive 3-4 sessions of this solution, followed by at least 20 sessions with 10% ethanol only. A minimum average of 0.3 g/kg ethanol consumption in 8 sessions prior to the beginning of any drug treatment is required.

### EXAMPLE 34

### Reduction of Nicotine Dependency

Biological material: Wistar-derived male rats (250-300 g) are housed in groups of two and maintained in a temperature-controlled environment on a 12 hour: 12 hour light cycle (0600h on-1800h off), upon arrival in the laboratory. Animals are given free access to food and water during a one-week habituation period to the laboratory. Animals used in the research studies are handled, housed, and sacrificed in accord with the current NIH guidelines regarding the use and care of laboratory animals, and all applicable local, state, and federal regulations and guidelines. Animals are handled daily for several days to desensitize them to handling stress before experimental testing. The cell sizes (n=8) provide reliable estimates of drug effects.

Drug Treatments: The Wistar-derived rats receive several doses of the test compound (0.00, 7.5, 10, and 15 mg/kg) administered intraperitonealy (i.p.), and a positive control compound, mecamylamine (1.5 mg/kg, subcutaneously (s.c.). The compounds are administered 30 minutes prior to SA sessions. The test compound is administered at 2 ml/kg for the 7.5 mg/kg (3.75 mg/ml) and 10 mg/kg (5 mg/ml), doses, and at 3 ml/kg for the 15 mg/kg dose (5 mg/ml). The compound is dissolved in corn oil (VEH), and sonicated for at least 30-minutes, up to 2 hours prior to administration. Mecamylamine is dissolved in 0.09% isotonic saline and administered at a volume of 1 ml/kg.

Apparatus: Food training and nicotine self-administration take place in 8 standard Coulbourn operant chambers. Each chamber is housed in a sound-attenuated box. Operant chambers are equipped with two levers, mounted 2 cm above the floor, and a cue light mounted 2 cm above the right lever on the back wall of the chamber. For food training, a food hopper is located 2-cm to the left/right of either lever, in the middle of the back wall. Intravenous infusions are delivered in a volume of 0.1 ml over a 1 second interval via an infusion pump (Razel, Conn.) housed outside of the sound attenuated chamber.

Food Training: Lever pressing is established as demonstrated by the method of Hyytia et al., (1996). Initially, rats are restricted to 15 grams of food daily (approximately 85% of their free-feeding body weight). After the second day of food restriction, rats are trained to respond for food under a fixed-ratio 1 (FR1) schedule of reinforcement (1 food pellet for each lever press) with a 1 second time-out (TO-1s) after each reinforcement. Training sessions are given twice per day, and TO periods are gradually increased to 20 seconds. Once rats obtain a steady baseline responding at a FR1-TO20s schedule of reinforcement, they are returned to ad libitum food prior to preparation for intravenous jugular catheter implant surgery.

Surgery: Rats are anesthetized with a ketamine/xylazine mixture and chronic silastic jugular catheters are inserted into the external jugular vein and passed subcutaneously to a polyethylene assembly mounted on the animal's back. The catheter assembly consists of a 13-cm length of silasitic tubing (inside diameter 0.31 mm; outside diameter 0.64 mm), attached to a guide cannula that is bent at a right angle. The cannula is embedded into a dental cement base and anchored with a 2×2 cm square of durable mesh. The catheter is passed subcutaneously from the rats back to the jugular vein where it is inserted and secured with a non-absorbable silk suture. Upon successful completion of surgery, rats are given 3-5 days to recover before self-administration sessions starts. During the recovery period, rats remain ad libitum food access, and have catheter lines flushed daily with 30 units/ml of heparinized saline containing 66 mg/ml of Timentin to prevent blood coagulation and infection in the catheters.

Nicotine Self-Administration: Following successful recovery from catheter implant surgery, rats are again food deprived to 85% of their free-feeding body weight. Once self-administration sessions begin, subjects are trained to IV self-administer nicotine in 1-hour baseline sessions, 5 days per week, under a FR1-TO-20 schedule of reinforcement until stable responding is achieved. Stable responding is defined as less than 20% variability across 3 consecutive sessions. After acquisition of stable responding for nicotine, various doses of the test compound is tested using a within-subjects Latin square design. Rats are allowed to self-administer nicotine after treatment with each dose of test compound for 1 test session, and subsequently “rebaselined” for 1-3 days before the next dose probe during one test self-administrations sessions. Following the testing of the first compound, rats receive the positive control compound, mecamylamine (1.5 mg/kg), administered according to a crossover design.

During SA sessions, rats are flushed with saline before test session to ensure catheter patency, and again flushed after test sessions with 30 units/ml of heparinized saline containing 66 mg/ml of Timentin, to prevent blood coagulation and infection in the catheters. If catheter patency is in question, demonstrated by an unexpected shift in response rates, or inability to draw blood from the catheter, 0.1 ml of a short-acting anesthetic (Brevital) is infused. Animals with patent catheters exhibit rapid loss of muscle tone within 3-seconds. Rats with catheters no longer patent according to the Brevital test are removed from the experiment.

Data Analysis

Data is collected on-line from multiple operant chambers, and reported as mean cumulative number of bar presses for nicotine. The data is analyzed using the StatView statistical package on a PC-compatible computer.

We claim:

1. A compound of Formula I: 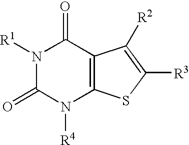
wherein
R¹ is hydrogen, optionally substituted C₁₋₄ alkyl, optionally substituted C₂₋₄ alkenyl, or optionally substituted C₂₋₄ alkynyl;
R² is hydrogen, optionally substituted C₁₋₄ alkyl, or a 5 or 6 membered optionally substituted monocyclic heterocycle containing 1, 2, 3, or 4 heteroatoms independently selected from oxygen, sulfur and nitrogen;
R³ is hydrogen, —C(O)NR⁵R⁶, in which R⁵ and R⁶ are independently selected from hydrogen, optionally substituted lower alkyl, or R³ is an optionally substituted 5 or 6 membered monocyclic heterocycle or heteroaryl containing 1, 2, 3, or 4 heteroatoms independently selected from oxygen, sulfur and nitrogen; or
R² and R³ taken together are an optionally substituted alkylene group which combine to form a 5 or 6 membered optionally substituted cyclic alkenyl ring, or a 5 or 6 membered heterocyclic ring in which the heteroatoms are oxygen or nitrogen; and
R⁴ is hydrogen or optionally substituted C₁₋₄ alkyl,
or a pharmaceutically acceptable salt, ester, prodrug, hydrate, or polymorph thereof.

2. The compound of claim 1, wherein R⁴ is hydrogen.

3. The compound of claim 2, wherein R² and R³ taken together combine to form a 5 or 6 membered optionally substituted cyclic alkenyl ring.

4. The compound of claim 3, wherein R¹ is ethyl, namely 3-ethyl-1,3,5,6,7,8-hexahydrobenzo[b]thiopheno[2,3-d]pyrimidine-2,4-dione.

5. The compound of claim 2, wherein R³ is hydrogen.

6. The compound of claim 5, selected from the group consisting of:
3-ethyl-5-methyl-1,3-dihydrothiopheno[2,3-d]pyrimidine-2,4-dione;
5-methyl-3-(2-methylpropyl)-1,3-dihydrothiopheno[2,3-d]pyrimidine-2,4-dione;
5-methyl-3-prop-2-enyl-1,3-dihydrothiopheno[2,3-d]pyrimidine-2,4-dione;
5-methyl-3-prop-2-ynyl-1,3-dihydrothiopheno[2,3-d]pyrimidine-2,4-dione;
3-ethyl-5-(2-furyl)-1,3-dihydrothiopheno[2,3-d]pyrimidine-2,4-dione;
3,5-dimethyl-1,3-dihydrothiopheno[2,3-d]pyrimidine-2,4-dione; and
3-ethyl-5-(5-methyl(2-furyl))-1,3-dihydrothiopheno[2,3-d]pyrimidine-2,4-dione.

7. The compound of claim 2, wherein R³ is an optionally substituted 5 or 6 membered monocyclic heterocycle or heteroaryl containing 1, 2, 3, or 4 heteroatoms independently selected from oxygen, sulfur and nitrogen.

8. The compound of claim 7, selected from the group consisting of:
3-ethyl-5-methyl-6-(1,3-oxazol-2-yl)-1,3-dihydrothiopheno[2,3-d]pyrimidine-2,4-dione;
6-(2-furyl)-5-methyl-3-(2-methylpropyl)-1,3-dihydrothiopheno[2,3-d]pyrimidine-2,4-dione;
3-ethyl-5-methyl-6-[3-benzyl(1,2,4-oxadiazol-5-yl)]-1,3-dihydrothiopheno[2,3-d]pyrimidine-2,4-dione;
3-ethyl-6-(2-furyl)-5-methyl-1,3-dihydrothiopheno[2,3-d]pyrimidine-2,4-dione;
3-ethyl-5-methyl-6-(2-thienyl)-1,3-dihydrothiopheno[2,3-d]pyrimidine-2,4-dione;
3-ethyl-6-(3-furyl)-5-methyl-1,3-dihydrothiopheno[2,3-d]pyrimidine-2,4-dione;
3-ethyl-5-methyl-6-[1-benzylpyrazol-4-yl]-1,3-dihydrothiopheno[2,3-d]pyrimidine-2,4-dione;
3-ethyl-5-methyl-6-(3-thienyl)-1,3-dihydrothiopheno[2,3-d]pyrimidine-2,4-dione;
5-methyl-3-(2-methylpropyl)-6-(1,3-oxazol-4-yl)-1,3-dihydrothiopheno[2,3-d]pyrimidine-2,4-dione;
3-ethyl-6-(6-methoxy(3-pyridyl))-5-methyl-1,3-dihydrothiopheno[2,3-d]pyrimidine-2,4-dione;
3-ethyl-6-(2-methoxy(3-pyridyl))-5-methyl-1,3-dihydrothiopheno[2,3-d]pyrimidine-2,4-dione;
6-(3-furyl)-5-methyl-3-(2-methylpropyl)-1,3-dihydrothiopheno[2,3-d]pyrimidine-2,4-dione;
3-ethyl-5-methyl-6-pyrrol-2-yl-1,3-dihydrothiopheno [2,3-d]pyrimidine-2,4-dione;
5-methyl-3-(2-methylpropyl)-6-(1,3-oxazol-2-yl)-1,3-dihydrothiopheno[2,3-d]pyrimidine-2,4-dione;
3-ethyl-5-methyl-6-(1,3-thiazol-2-yl)-1,3-dihydrothiopheno[2,3-d]pyrimidine-2,4-dione;
3-(cyclopropylmethyl)-5-methyl-6-(2-furyl)-1,3-dihydrothiopheno [2,3-d]pyrimidine-2,4-dione;
6-(2-furyl)-3,5-dimethyl-1,3-dihydrothiopheno[2,3-d]pyrimidine-2,4-dione;
3-(cyclopropylmethyl)-5-methyl-6-(2-thienyl)-1,3-dihydrothiopheno[2,3-d]pyrimidine-2,4-dione;
3,5-dimethyl-6-(2-thienyl)-1,3-dihydrothiopheno[2,3-d]pyrimidine-2,4-dione;
3-ethyl-5-methyl-6-(3-methyl(2-thienyl))-1,3-dihydrothiopheno[2,3-d]pyrimidine-2,4-dione;
3-ethyl-5-methyl-6-(4-methyl(2-thienyl))-1,3-dihydrothiopheno [2,3-d]pyrimidine-2,4-dione;
3-ethyl-5-methyl-6-(2-pyridyl)-1,3-dihydrothiopheno[2,3-d]pyrimidine-2,4-dione;
1-ethyl-6-(2-furyl)-5-methyl-3-(2-methylpropyl)-1,3-dihydrothiopheno[2,3-d]pyrimidine-2,4-dione;
6-((4S)-4-methyl(1,3-oxazolin-2-yl))-5-methyl-3-propyl-1,3-dihydrothiopheno[2,3-d]pyrimidine-2,4-dione;
6-((4R)-4-methyl(1,3-oxazolin-2-yl))-5-methyl-3-propyl-1,3-dihydrothiopheno[2,3-d]pyrimidine-2,4-dione;
3-ethyl-5-methyl-6-(1,3-oxazolin-2-yl)-1,3-dihydrothiopheno[2,3-d]pyrimidine-2,4-dione;
6-((4S)-4-methyl(1,3-oxazolin-2-yl))-5-methyl-3-(2-methylpropyl)-1,3-dihydrothiopheno[2,3-d]pyrimidine-2,4-dione;
6-((4S)-4-ethyl(1,3-oxazolin-2-yl))-5-methyl-3-(2-methylpropyl)-1,3-dihydrothiopheno[2,3-d]pyrimidine-2,4-dione;
6-((5R)-5-methyl(1,3-oxazolin-2-yl))-5-methyl-3-(2-methylpropyl)-1,3-dihydrothiopheno[2,3-d]pyrimidine-2,4-dione; and
5-methyl-3-(2-methylpropyl)-6-(1,3-oxazolin-2-yl)-1,3-dihydrothiopheno[2,3-d]pyrimidine-2,4-dione.

9. The compound of claim 2, wherein R³ is C(O)NR⁵R⁶.

10. The compound of claim 9, selected from the group consisting of:
(3-ethyl-5-methyl-2,4-dioxo(1,3-dihydrothiopheno[2,3-d]pyrimidin-6-yl))-N-methylcarboxamide;
N-methyl(5-methyl-2,4-dioxo-3-propyl(1,3-dihydrothiopheno[2,3-d]pyrimidin-6-yl))carboxamide;
N-((1R)-2-hydroxy-isopropyl)(3-ethyl-5-methyl-2,4-dioxo(1,3-dihydrothiopheno[2,3-d]pyrimidin-6-yl))carboxamide; and
(3,5-dimethyl-2,4-dioxo(1,3-dihydrothiopheno[2,3-d]pyrimidin-6-yl))-N-Methylcarboxamide.

11. The compound of claim 1, wherein R⁴ is optionally substituted C₁₋₄ alkyl.

12. The compound of claim 11, wherein R² is methyl.

13. The compound of claim 11, wherein R³ is hydrogen.

14. The compound of claim 13, selected from the group consisting of:
1,5-dimethyl-3-prop-2-ynyl-1,3-dihydrothiopheno[2,3-d]pyrimidine-2,4-dione; and
3-ethyl-1-methyl-5-(5-methyl(2-furyl))-1,3-dihydrothiopheno[2,3-d]pyrimidine-2,4-dione.

15. The compound of claim 12, wherein R³ is an optionally substituted 5 or 6 membered monocyclic heterocycle or heteroaryl containing 1, 2, 3, or 4 heteroatoms independently selected from oxygen, sulfur and nitrogen.

16. The compound of claim 15, selected from the group consisting of:
6-((5R)-5-methyl(1,3-oxazolin-2-yl))-1,5-dimethyl-3-(2-methylpropyl)-1,3-dihydrothiopheno[2,3-d]pyrimidine-2,4-dione;
6-((4S)-4-ethyl(1,3-oxazolin-2-yl))-1,5-dimethyl-3-(2-methylpropyl)-1,3-dihydrothiopheno[2,3-d]pyrimidine-2,4-dione;
6-((4S)-4-methyl(1,3-oxazolin-2-yl))-1,5-dimethyl-3-(2-methylpropyl)-1,3-dihydrothiopheno[2,3-d]pyrimidine-2,4-dione;
3-ethyl-6-(2-furyl)-1,5-dimethyl-1,3-dihydrothiopheno[2,3-d]pyrimidine-2,4-dione;
3-ethyl-6-(3-furyl)-1,5-dimethyl-1,3-dihydrothiopheno[2,3-d]pyrimidine-2,4-dione;
3-ethyl-1,5-dimethyl-6-(4-methyl(2-thienyl))-1,3-dihydrothiopheno[2,3-d]pyrimidine-2,4-dione;
1,5-dimethyl-3-(2-methylpropyl)-6-(1,3-oxazol-2-yl)-1,3-dihydrothiopheno[2,3-d]pyrimidine-2,4-dione;
3-ethyl-1,5-dimethyl-6-(1,3-thiazol-2-yl)-1,3-dihydrothiopheno[2,3-d]pyrimidine-2,4-dione;
3-ethyl-1,5-dimethyl-6-pyrrol-2-yl-1,3-dihydrothiopheno[2,3-d]pyrimidine-2,4-dione;
1,5-dimethyl-3-(2-propylmethyl)-6-(1,3-oxazol-4-yl)-1,3-dihydrothiopheno[1,3-d]pyrimidine-2,4-dione; and
3-ethyl-1,5-dimethyl-6-(2-pyridyl)-1,3-dihydrothiopheno[2,3-d]pyrimidine-2,4-dione.

17. A pharmaceutical composition comprising a therapeutically effective amount of the compound of claim 1 or a pharmaceutically acceptable salt, ester, prodrug, hydrate, or polymorph thereof.

18. A method for treating a disease or condition in a mammal that can be treated with an A_{2A} receptor antagonist compound comprising administering to a mammal in need thereof a therapeutically effective dose of a compound of claim 1 or a pharmaceutically acceptable salt, ester, prodrug, hydrate, or polymorph thereof.

19. The method of claim 18, wherein the disease state is selected from the group consisting of obesity, CNS disorders (including the “movement disorders” Parkinson's disease, Huntington's Chorea, and catalepsy), cerebral ischemia, excitotoxicity, cognitive and physiological disorders, depression, ADHD, and drug addiction (including alcohol, amphetamine, cannabinoid, cocaine, nicotine, and opioid additction).

20. A method for inhibiting coronary vasodilation to prevent coronary steal in a mammal, comprising administering to a mammal in need thereof a therapeutically effective dose of a compound of claim 1.

21. A compound of Formula II: 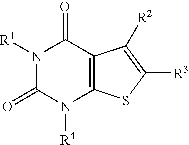
wherein
R¹ is hydrogen, optionally substituted C₁₋₄ alkyl, optionally substituted C₂₋₄ alkenyl, or optionally substituted C₂₋₄ alkynyl;
R² is hydrogen, optionally substituted C₁₋₄ alkyl, or a 5 or 6 membered optionally substituted monocyclic heterocycle containing 1, 2, 3, or 4 heteroatoms independently selected from oxygen, sulfur and nitrogen;
R³ is —COOR⁷, —C(O)R⁸, or optionally substituted lower alkyl, in which R⁷ is hydrogen or optionally substituted lower alkyl, and R⁸ is hydrogen or optionally substituted lower alkyl; and
R⁴ is hydrogen or optionally substituted C₁₋₄ alkyl.

22. The compound of claim 21, selected from the group consisting of:
3-ethyl-5,6-dimethyl-1,3-dihydrothiopheno[2,3-d]pyrimidine-2,4-dione;
ethyl3,5-dimethyl-2,4-dioxo-1,3-dihydrothiopheno[2,3-d]pyrimidine-6-carboxylate;
5-methyl-2,4-dioxo-3-propyl-1,3-dihydrothiopheno[2,3-d]pyrimidine-6-carboxylic acid;
ethyl5-methyl-2,4-dioxo-3-prop-2-ynyl-1,3-dihydrothiopheno[2,3-d]pyrimidine-6-carboxylate; and
ethyl1,5-dimethyl-2,4-dioxo-3-prop-2-ynyl-1,3-dihydrothiopheno[2,3-d]pyrimidine-6-carboxylate.
